(19) United States
(12) Patent Application Publication
(10) Pub. No.: US 2023/0049760 A1
(43) Pub. Date: Feb. 16, 2023

(54) LIPASE-MODIFIED STRAIN

(71) Applicant: DSM IP ASSETS B.V., Heerlen (NL)

(72) Inventors: Jenna MCMAHON, Tewksbury, MA (US); Elvin Irsan KOOI, Leiderdorp (NL); Liang WU, Delft (NL); René Marcel DE JONG, Amsterdam (NL); Valmik Kanubhai VYAS, Winchester, MA (US); Peter Louis HOUSTON, Boston, MA (US)

(21) Appl. No.: 17/789,598

(22) PCT Filed: Dec. 18, 2020
(86) PCT No.: PCT/EP2020/087018

§ 371 Date: Jun. 28, 2022

(30) Foreign Application Priority Data
Dec. 30, 2019 (CH) 01716/19
Jul. 27, 2020 (EP) 20187829.5

(51) Int. Cl. C12N 9/20; C12P 23/00; C12Q 1/44

(52) U.S. Cl. CPC C12N 9/20; C12P 23/00; C12Q 1/44; G01N 2333/92

(57) ABSTRACT

The present invention is related to a retinoid-producing host cell, particularly oleaginous yeast, modified such that the percentage of retinyl acetate based on the total retinoids produced by such host cell is increased during fermentation using triglyceride oils, like for example vegetable oil, as carbon source, wherein the activity of certain endogenous hydrolases or transferases involved in undesired conversions of retinol or retinol acetate is reduced or abolished. Particularly, such modified host cell might be useful in a biotechnological process for production of vitamin A.

The present invention is related to a retinoid-producing host cell, particularly oleaginous yeast, modified such that the percentage of retinyl acetate based on the total retinoids produced by such host cell is increased during fermentation using triglyceride oils, like for example vegetable oil, as carbon source, wherein the activity of certain endogenous hydrolases or transferases involved in undesired conversions of retinol or retinol acetate is reduced or abolished. Particularly, such modified host cell might be useful in a biotechnological process for production of vitamin A.

Retinoids, including vitamin A, are one of very important and indispensable nutrient factors for human beings which must be supplied via nutrition. Retinoids promote well-being of humans, inter alia in respect of vision, the immune system and growth. Retinyl acetate is an important intermediate or precursor in the process of vitamin A production.

Current chemical production methods for retinoids, including vitamin A and precursors thereof, have some undesirable characteristics such as e.g. high-energy consumption, complicated purification steps and/or undesirable by-products. Therefore, over the past decades, other approaches to manufacture retinoids, including vitamin A and precursors thereof, comprising microbial conversion steps have been investigated, which would lead to more economical as well as ecological vitamin A production.

In general, the biological systems that produce retinoids are industrially intractable and/or produce the compounds at such low levels that commercial scale isolation is not practical. The most limiting factors include instability of intermediates in such biological systems and/or the relatively high production of by-products, such as e.g. retinyl fatty esters, particularly using oleaginous host cells grown on vegetable oils as carbon source.

WO2019/058000 describes a novel fermentative process from beta-carotene towards retinol and retinyl acetate, an intermediate that is deemed more stable than retinol, using a carotenoid-producing host cell grown on corn oil, said host cell expressing heterologous beta-carotene oxidase (BCO), retinal reductase (RDH), and acetyl-transferase (ATF). However, a relatively high percentage of retinol produced by such oleaginous host cell is “lost” for vitamin A production, i.e. converted into undesired by-products catalyzed by endogenous hydrolases and/or transferases of the host cell.

Thus, it is an ongoing task to improve the product-specificity and/or productivity of fermentative processes towards conversion of retinol into retinyl acetate, a stable intermediate in vitamin A production. Particularly, it is desirable to develop a fermentative process using preferably oleaginous host cells growing on vegetable oil with limited formation of by-products and maximal accumulation of retinyl acetate without compromising the growth of the host cell.

Surprisingly, we now found that modification of the host cell, particularly oleaginous yeast, i.e. modification, particularly blockage, of certain enzymes involved in pre-digestion of vegetable oil into glycerol and fatty acids could lead to an increase in retinyl acetate formation, i.e. percentage of retinyl acetate based on total retinoids might be increased by at least about 30%, such as to percentage in the range of about 70-90% and more, compared to a process using the respective non-modified host cell.

Particularly, the present invention is directed to a retinoid-producing host cell capable of retinyl acetate formation, such as a fungal host cell, preferably oleaginous yeast cell such as e.g. Yarrowia, comprising one or more genetic modification(s), i.e. reduction or abolishment, preferably abolishment, of certain endogenous genes encoding hydrolase or transferase enzymes, particularly including e.g. genes encoding endogenous lipases and/or esterases, including but not limited to modification in the activity of an endogenous gene with at least about 50%, such as 60, 70, 80, 90, 95, 98, or 100% identity to SEQ ID NO:5, wherein SEQ ID NO:5 corresponds to LIP8 obtainable from Yarrowia lipolytica.

In one aspect, the present invention is directed to a fermentation process using such modified host cell defined herein said host cell being grown on triglyceride oils, like for example vegetable oil, such as e.g. corn oil, as carbon source, wherein the formation of retinyl acetate from conversion of retinol is increased, resulting in a percentage of about at least 70%, such as e.g. about 75, 80, 85, 90, 95, 98% or more, including 100%, retinyl acetate based on total retinoids present in/produced by said modified host cell.

Suitable endogenous hydrolases or transferases to be modified according to the present invention might be selected from enzymes with lipase and/or esterase activity. The term “lipase” is used interchangeably herein with the term “esterase” or “enzyme having lipase and/or esterase activity”. It refers to enzymes involved in pre-digestion of triglyceride oils such as e.g. vegetable oil into glycerol and fatty acids that are normally expressed in oleaginous host cells. Suitable enzymes to be modified in a host cell as defined herein might be selected from endogenous enzymes belonging to EC class 3.1.1.-, including, but not limited to one or more enzyme(s) with activities corresponding to Yarrowia LIP2, LIP3, LIP8, TGL1, LIP16, LIP17, LIP18, or LIP4 activities.

As used herein, an enzyme having activity corresponding to the respective LIP activity in Yarrowia includes not only the genes originating from Yarrowia, e.g. Yarrowia lipolytica, such as e.g. Yarrowia LIP2, LIP3, LIP8, TGL-1, LIP16, LIP17, LIP18, LIP4 or combinations thereof, but also includes enzymes having equivalent enzymatic activity but are originated from another source organism, particularly retinyl acetate-producing oleaginous host cell, wherein a modification of such equivalent endogenous genes would lead to an increase in retinol to retinyl acetate conversion as defined herein.

The present invention is directed to a host cell which is modified in certain endogenous hydrolase/transferase activities leading to an increase in retinyl acetate in a vitamin A fermentation process as defined herein. Suitable host cells to be modified are selected from retinoid-producing host cells, particularly retinyl acetate-producing host cells, wherein retinyl acetate is formed via enzymatic conversion of retinol catalyzed by acetylating enzymes (ATFs), e.g. fungal host cells including oleaginous yeast cells, such as e.g. Rhodosporidium, Lipomyces or Yarrowia, preferably Yarrowia, more preferably Yarrowia lipolytica, wherein the conversion of retinol into retinyl acetate is enhanced leading to a percentage of retinyl acetate based on total retinoids in the cell which is increased by at least about 10% via modification of said endogenous enzyme activity, such as lipase and/or esterase activities, as defined herein, and wherein the modification comprises genetic modification, such as e.g. reducing/abolishing activity of endogenous genes encoding certain Yarrowia lipases/esterases or corresponding endogenous enzyme activities from other oleaginous host cells as specified herein, including but not limited to deletion of the corresponding genes.

As defined herein, a “modified host cell” is compared to a “wild-type host cell”, i.e., the respective host cell without such modification in the defined enzyme activities, i.e. wherein said corresponding endogenous enzyme is (still) expressed and active in vivo.

In one embodiment, the present invention provides a modified host cell, such as modified retinyl acetate-producing oleaginous host cell, comprising a modification in a polypeptide with at least about 50%, such as 60, 70, 80, 90, 95, 98, or 100% identity to SEQ ID NO:5, including but not limited to LIP8 obtainable from Yarrowia lipolytica, wherein the activity of said polypeptide is reduced or abolished, preferably abolished, including reduction or abolishment of gene expression, wherein the use of such modified host cell in a fermentation in the presence of triglyceride oils, such as e.g. vegetable corn oil, as carbon source results in increased percentage of retinyl acetate from conversion of retinol, such as at least about 70% retinyl acetate based on total retinoids present in the respective host cell as defined herein. Particularly, the host cell is selected from Yarrowia, such as Yarrowia lipolytica, wherein the activity of LIP8 according to SEQ ID NO:5, including a polypeptide encoded by a polynucleotide according to SEQ ID NO:6, is reduced or abolished, preferably abolished, leading to about 30% or more retinyl acetate based on total retinoids in the host cell. LIP8 according to SEQ ID NO:5 is derived from RefSeq YALI0_B09361g. Reduction or abolishment of LIP8 or a corresponding enzyme from another oleaginous yeast as defined herein might be combined with reduction or abolishment of further endogenous enzymes including but not limited to enzymes with activities equivalent to Yarrowia LIP2, LIP3, TGL1, LIP16, LIP17, LIP18, or LIP4 activities, including enzymes with at least about 50% identity to a sequence selected from the group consisting of SEQ ID NO:1, 3, 7, 9, 11, 13, 15 and combinations thereof.

In one embodiment, the present invention provides a modified host cell, such as modified retinyl acetate-producing oleaginous host cell, comprising a modification in a polypeptide with at least about 50%, such as 60, 70, 80, 90, 95, 98, or 100% identity to SEQ ID NO:1, including but not limited to LIP2 obtainable from Yarrowia lipolytica, wherein the activity of said polypeptide is reduced or abolished, preferably abolished, including reduction or abolishment of gene expression, wherein the use of such modified host cell in a fermentation in the presence of triglyceride oils, such as e.g. vegetable corn oil, as carbon source results in increased percentage of retinyl acetate from conversion of retinol, such as at least about 70% retinyl acetate based on total retinoids present in the respective host cell as defined herein. Particularly, the host cell is selected from Yarrowia, such as Yarrowia lipolytica, wherein the activity of LIP2 according to SEQ ID NO:1, including a polypeptide encoded by a polynucleotide according to SEQ ID NO:2, is reduced or abolished, preferably abolished, leading to about 30% or more retinyl acetate based on total retinoids in the host cell. LIP2 according to SEQ ID NO:1 is derived from RefSeq YALI0_A20350g. Reduction or abolishment of LIP2 or a corresponding enzyme from another oleaginous yeast as defined herein might be combined with reduction or abolishment of further endogenous enzymes including but not limited to enzymes with activities equivalent to Yarrowia LIP8, LIP3, TGL1, LIP16, LIP17, LIP18, or LIP4 activities, including enzymes with at least about 50% identity to a sequence selected from the group consisting of SEQ ID NO:5, 3, 7, 9, 11, 13, 15, and combinations thereof.

In a further embodiment, the present invention provides a modified host cell, such as modified retinyl acetate-producing oleaginous host cell, comprising a modification in a polypeptide with at least about 50%, such as 60, 70, 80, 90, 95, 98, or 100% identity to SEQ ID NO:3, including but not limited to LIP3 obtainable from Yarrowia lipolytica, wherein the activity of said polypeptide is reduced or abolished, preferably abolished, including reduction or abolishment of gene expression, wherein the use of such modified host cell in a fermentation in the presence of triglyceride oils, such as e.g. vegetable corn oil, as carbon source results in increased percentage of retinyl acetate from conversion of retinol, such as at least about 70% retinyl acetate based on total retinoids present in the respective host cell as defined herein. Particularly, the host cell is selected from Yarrowia, such as Yarrowia lipolytica, wherein the activity of LIP3 according to SEQ ID NO:3, including a polypeptide encoded by a polynucleotide according to SEQ ID NO:4, is reduced or abolished, preferably abolished, leading to about 30% or more retinyl acetate based on total retinoids in the host cell. LIP3 according to SEQ ID NO:3 is derived from RefSeq YALI0_B08030g. Reduction or abolishment of LIP3 or a corresponding enzyme from another oleaginous yeast as defined herein might be combined with reduction or abolishment of further endogenous enzymes including but not limited to enzymes with activities equivalent to Yarrowia LIP8, LIP2, TGL1, LIP16, LIP17, LIP18, or LIP4 activities, including enzymes with at least about 50% identity to a sequence selected from the group consisting of SEQ ID NO:5, 1, 7, 9, 11, 13, 15 and combinations thereof.

In a further embodiment, the present invention provides a modified host cell, such as modified retinyl acetate-producing oleaginous host cell, comprising a modification in a polypeptide with at least about 50%, such as 60, 70, 80, 90, 95, 98, or 100% identity to SEQ ID NO:15, including but not limited to LIP4 obtainable from Yarrowia lipolytica, wherein the activity of said polypeptide is reduced or abolished, preferably abolished, including reduction or abolishment of gene expression, wherein the use of such modified host cell in a fermentation in the presence of triglyceride oils, such as e.g. vegetable corn oil, as carbon source results in increased percentage of retinyl acetate from conversion of retinol, such as at least about 70% retinyl acetate based on total retinoids present in the respective host cell as defined herein. Particularly, the host cell is selected from Yarrowia, such as Yarrowia lipolytica, wherein the activity of LIP4 according to SEQ ID NO:15, including a polypeptide encoded by a polynucleotide according to SEQ ID NO:16, is reduced or abolished, preferably abolished, leading to about 30% or more retinyl acetate based on total retinoids in the host cell. LIP4 according to SEQ ID NO:15 is derived from RefSeq YALI0_E08492g. Reduction or abolishment of LIP4 or a corresponding enzyme from another oleaginous yeast as defined herein might be combined with reduction or abolishment of further endogenous enzymes including but not limited to enzymes with activities equivalent to Yarrowia LIP8, LIP2, LIP3, TGL1, LIP16, LIP17, or LIP18 activities, including enzymes with at least about 50% identity to a sequence selected from the group consisting of SEQ ID NO:5, 1, 3, 7, 9, 11, 13 and combinations thereof.

According to further embodiments, the present invention provides a modified host cell, such as modified retinyl acetate-producing oleaginous host cell, comprising a modification in a polypeptide selected from the group consisting of polypeptides with at least about 50%, such as 60, 70, 80, 90, 95, 98, or 100% identity to SEQ ID NO:7, 9, 11, 13, and combinations thereof; including but not limited to an enzyme obtainable from Yarrowia lipolytica selected from the group consisting of TGL1, LIP16, LIP17, LIP18, and combinations thereof; wherein the activity of said polypeptide(s) is reduced or abolished, preferably abolished, including reduction or abolishment of gene expression, wherein the use of such modified host cell in a fermentation in the presence of triglyceride oils, such as e.g. vegetable corn oil, as carbon source results in increased percentage of retinyl acetate from conversion of retinol, such as at least about 70% retinyl acetate based on total retinoids present in the respective host cell as defined herein. Particularly, the host cell is selected from Yarrowia, such as Yarrowia lipolytica, wherein the activity of an enzyme selected from TGL1, LIP16, LIP17, LIP18 or combinations thereof according to SEQ ID NO:7, 9, 11, 13, including polypeptide(s) encoded by polynucleotide(s) according to SEQ ID NO:8, 10, 12, 14 is reduced or abolished, preferably abolished, leading to about 30% or more retinyl acetate based on total retinoids in the host cell. TGL1 according to SEQ ID NO:7 is derived from RefSeq YALI0_E32035g. LIP16 according to SEQ ID NO:9 is derived from RefSeq YALI0_D18480g. LIP17 according to SEQ ID NO:11 is derived from RefSeq YALI0_F32131g. LIP18 according to SEQ ID NO:13 is derived from RefSeq YALI0_B20350g. Reduction or abolishment of an enzyme selected from the group consisting of TGL1, LIP16, LIP17, LIP18, and combinations thereof or a corresponding enzyme from another oleaginous yeast as defined herein might be combined with reduction or abolishment of further endogenous enzymes including but not limited to enzymes with activities equivalent to Yarrowia LIP8, LIP2 and/or LIP3 and/or LIP4 activities, including enzymes with at least about 50% identity to a sequence selected from the group consisting of SEQ ID NO:5, 1, 3, 15 and combinations thereof.

Preferably, a modified host cell according to the present invention comprises a modification in an enzyme with activity of an enzyme with at least about 50% identity to LIP8 according to SEQ ID NO:5 such as obtainable from Yarrowia or an enzyme from another host cell with activity equivalent to Yarrowia LIP8 as defined herein, leading to a percentage of retinyl acetate based on total retinoids in the range of about 70-90% or more, such as e.g. in a process wherein the host cell is grown in the presence of triglyceride oils, such as e.g. vegetable corn oil, as carbon source. The percentage of retinyl acetate might be furthermore increased, such as e.g. by at least about 10% based on total retinoids, such as e.g. in a process wherein the host cell is grown in the presence of triglyceride oils, such as e.g. vegetable corn oil, as carbon source, with combination of further modifications in the endogenous enzyme activity in the host cell. Particularly preferred are combination with further modifications, such as e.g. modification in the activity of an enzyme with at least about 50% identity to LIP2 and/or LIP3 and/or LIP4 according to SEQ ID NO:1 or 3 or 15 such as obtainable from Yarrowia or enzymes from another host cell with activities equivalent to Yarrowia LIP2 and/or LIP3 and/or LIP4. Further increase in retinyl acetate percentage based on total retinoids might be possible via introduction of one or more modifications in the activity of one or more enzyme(s) with at least about 50% identity to an enzyme selected from the group consisting of TGL1, LIP16, LIP17, LIP18 and combinations thereof according to SEQ ID NO:7, 9, 11, 13 such as obtainable from Yarrowia or enzymes from another host cell with activities equivalent to an enzyme selected for the group consisting of Yarrowia TGL1, LIP16, LIP17, and LIP18.

As used herein, “activity” of an enzyme, particularly hydrolase or transferase activity, including activity of lipases or esterases as defined herein, is defined as “specific activity” i.e. its catalytic activity, i.e. its ability to catalyze formation of a product from a given substrate, such as e.g. the formation of retinyl fatty esters. An enzyme, e.g. a lipase or esterase, is active, if it performs its catalytic activity in vivo, i.e. within the host cell as defined herein or within a system in the presence of a suitable substrate. The skilled person knows how to measure enzyme activity, in particular activity of lipases as defined herein, including but not limited to enzyme with activities corresponding to Yarrowia LIP2 and/or LIP3 and/or LIP8 and/or TGL1 and/or LIP16 and/or LIP17 and/or LIP18 and/or LIP4 activity. Analytical methods to evaluate the capability of lipases/esterases as defined herein involved in formation of retinyl fatty esters are known in the art and include measurement via HPLC and the like. With regards to activity of LIP2, LIP3, LIP4, LIP8, TGL1, LIP16, LIP17 and/or LIP18 as defined herein, the skilled person might measure the formation of retinyl fatty esters from conversion of retinol in comparison to the formation of retinyl acetate from conversion of retinol, both measured with a modified and wild-type host cell.

As used herein, an enzyme, particularly a lipase or esterase as defined herein, having “reduced or abolished” activity means a decrease in its specific activity, i.e. reduced/abolished ability to catalyze formation of a product from a given substrate, such as conversion of triglycerides, such as e.g. vegetable oil, preferably corn oil, into glycerol and fatty acids during fermentation, including reduced or abolished activity of the respective (endogenous) gene encoding such lipases or esterases. A reduction by 100% is referred herein as abolishment of enzyme activity, achievable e.g. via deletion, insertions, frameshift mutations, missense mutations or premature stop-codons in the endogenous gene encoding said enzyme or blocking of the expression and/or activity of said endogenous gene(s) with known methods.

As used herein, “deletion” of a gene leading to abolishment of gene activity includes all mutations in the nucleic acid sequence that can result in an allele of diminished function, including, but not limited to deletions, insertions, frameshift mutations, missense mutations, and premature stop codons, wherein deleted means that the corresponding gene/protein activity, such as particularly endogenous lipase activity, cannot be detected (any more) in the host cell.

In one particular embodiment, the present invention is directed to a modified host cell as defined herein capable of retinyl acetate formation, wherein formation of retinyl acetate is increased during fermentation compared to the formation of retinyl acetate using the respective non-modified host cell. As used herein, increased retinyl acetate formation means a percentage of at least about 30%, such as e.g. about 75, 80, 85, 90, 95, 98% or more, including 100%, retinyl acetate based on total retinoids present in/produced by said modified host cell.

Thus, the present invention is directed to a retinoid-producing modified host cell, particularly retinyl acetate-producing fungal host cell, wherein the percentage of retinyl acetate based on the total amount of retinoids produced by said host cell is at least in the range of about 70-90%, such as at least about 70%, such as e.g. about 75, 80, 85, 90, 95, 98% or more, including 100%, as compared to the respective non-modified host cell, and wherein said modification means reduction or abolishment of endogenous lipase or esterase activities, including but not limited to activity corresponding to Yarrowia LIP8 and optionally furthermore to activity corresponding to Yarrowia LIP2 and/or LIP3 and/or LIP4 and/or TGL1 and/or LIP16 and/or LIP17 and/or LIP18.

The host cell to be modified according to the present invention might be selected from Yarrowia lipolytica as disclosed in WO2019/058001 or WO2019/057999, wherein the formation of retinyl acetate from beta-carotene is optimized via heterologous expression of beta-carotene oxidases (BCO), retinol dehydrogenase (RDH) and/or acetyl-transferases (ATF). Particularly, a modified host cell as defined herein might be expressing a BCO originated from Drosophila melanogaster, RDH originated from Fusarium fujikuroi, and fungal ATF, such as e.g. ATF originated from Lachancea or Saccharomyces. To enhance the conversion of beta-carotene into retinal into retinol into retinyl acetate produced by the host cell, said enzymes might comprise one or more mutations leading to improved acetylation of retinol into retinyl acetate.

Introduction of modification(s) in the retinoid-producing host cell in order to produce less or no copies of genes and/or proteins, such as lipases or esterases and respective genes as defined herein, including generation of modified suitable host cell capable of retinyl acetate formation as defined herein with reduced/abolished activity in enzymes corresponding to Yarrowia LIP8, optionally further comprising reduced/abolished activity in enzyme(s) corresponding to Yarrowia LIP2 and/or LIP3 and/or LIP4 and/or TGL1 and/or LIP16 and/or LIP17 and/or LIP18 may include the use of weak promoters, or the introduction of one or more mutations) (e.g. insertion, deletion/knocking-out or point/frameshift/missense mutation, premature stop-codons) of (parts of) the respective enzymes (as described herein), in particular its regulatory elements, leading to reduction/abolishment of said enzyme activity, such as e.g. inactivation via in vivo mutagenesis, for example by mutation of the catalytic residues or by making mutations or deletions that interfere with protein folding or pre- or pro-sequence cleavage needed to activate the lipase/esterase upon secretion by the host cell. The skilled person knows how to genetically manipulate or modify a host cell as defined herein resulting in reduction/abolishment of such activity, e.g. hydrolase/transferase activity, including lipase or esterase activity, as defined herein. These genetic manipulations include, but are not limited to, e.g. gene replacement, gene amplification, gene disruption, transfection, transformation using plasmids, viruses, or other vectors. An example of such a genetic manipulation may for instance affect the interaction with DNA that is mediated by the N-terminal region of enzymes as defined herein or interaction with other effector molecules. In particular, modifications leading to reduced/abolished specific enzyme activity may be carried out in functional, such as functional for the catalytic activity, parts of the proteins. Furthermore, reduction/abolishment of enzyme specific activity might be achieved by contacting said enzymes with specific inhibitors or other substances that specifically interact with them. In order to identify such inhibitors, the respective enzymes, such as e.g. certain lipases as defined herein, may be expressed and tested for activity in the presence of compounds suspected to inhibit their activity.

The generation of a mutation into nucleic acids or amino acids, i.e. mutagenesis, may be performed in different ways, such as for instance by random or side-directed mutagenesis, physical damage caused by agents such as for instance radiation, chemical treatment, or insertion of a genetic element. The skilled person knows how to introduce mutations.

A modified host cell capable of retinyl acetate production according to the present invention might comprise further modifications including reduction or abolishment of further lipase or esterase activities present in said host cell as long as they result in increasing the percentage of retinyl acetate based on the total retinoids produced in fermentation as defined herein without compromising the growth of such modified host cell.

Thus, the present invention furthermore includes a process for identification of endogenous hydrolases to be modified, such as e.g. via reduction or abolishment of the specific enzyme activity, including lipases/esterases with activities corresponding to Yarrowia LIP8 and/or LIP2 and/or LIP3 and/or LIP4 and/or TGL and/or LIP16 and/or LIP17 and/or LIP18, comprising the step of over-expressing the respective endogenous genes one by one in a suitable host cell, such as e.g. retinyl-acetate-producing host cell, to see if that amplifies a negative effect, like decreasing the percentage of retinyl acetate. Subsequently, one can reduce/abolish, e.g. inactivate the corresponding genes such as e.g. via deletion, the activity of those enzymes for which this over-expression leads to reduction in retinyl acetate during fermentation of said host cell, and picking the clones with increased retinyl acetate formation.

A particular embodiment is directed to a process for the identification of suitable endogenous hydrolases/transferase as defined herein and to be modified according to the present invention, comprising the steps of: pre-digestion of vegetable oil into glycerol and fatty acids,

(2) selection of endogenous lipase or esterase enzymes based on sequence homology of at least about 50%, such as e.g. 60, 70, 80, 90, 95, 98 or 100% to SEQ ID NO:1, 3, 5, 7, 9, 11, 13, 15

(3) overexpression of selected genes and comparison of retinyl acetate percentage based on total retinoids,

(4) selection of genes, wherein overexpression had a negative impact on retinyl acetate percentage in the retinoid mix, and

(5) reduction or abolishment, e.g. inactivation, such as e.g. via deletion, of selected genes which upon overexpression had a negative impact on retinyl acetate formation. According to one specific aspect of the present invention, the modified host cell as defined herein might be used in a process for reducing the formation of by-products in vitamin A fermentation process with increasing the percentage of retinyl acetate present in a retinoid mix produced by the host cell. The modified host cell as defined herein might comprise further modifications, including the introduction (and expression) of host-optimized heterologous polynucleotides. The skilled person knows how to generate such modified polynucleotides. It is understood that such host-optimized nucleic acid molecules as well as molecules comprising so-called silent mutations are included by the present invention as long as they still result in modified host cells carrying modified lipase/esterase activity as defined herein.

The terms “sequence identity”, “% identity” or “sequence homology” are used interchangeable herein. For the purpose of this invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids, they are identical or have 100% identity. With regards to enzymes originated from plants, the skilled person knows plant-derived enzymes might contain a chloroplast targeting signal which is to be cleaved via specific enzymes, such as e.g. chloroplast processing enzymes (CPEs).

In one embodiment, the present invention features the use of a modified host cell as defined herein in a fermentation process for production of retinol and retinyl acetate, comprising the step of enzymatic conversion of retinal, particularly with a percentage of at least about 65-90% trans-retinal based on the total amount of retinoids produced by such host cell, via action of suitable retinol dehydrogenases (RDHs), as e.g. exemplified in WO2019/057998.

Optionally, the retinol is isolated and/or further purified from the fermentation medium. Such process might comprise further steps, such as e.g. enzymatic conversion of beta-carotene into retinal via action of suitable BCOs, preferably BCOs with a selectivity towards formation of trans-retinal, more preferably leading to at least about 65-90% trans-isoforms based on the total amount of retinoids produced by said host cell, such as e.g. exemplified in WO2019/057999. Thus, a preferred process for production of retinol and/or retinyl acetate using a modified host cell as defined herein comprises the steps of (1) enzymatic conversion of beta-carotene into retinal via action of suitable BCOs, (2) enzymatic conversion of retinal into retinol via action of suitable RDHs, and optionally (3) isolation and/or purification of retinol from the fermentation medium.

In one embodiment, the present invention features the use of a modified host cell as defined herein in a fermentation process for production of retinyl acetate, comprising the step of enzymatic conversion of retinol via action of suitable acetyl transferases (ATFs), as e.g. exemplified in WO2019/058001. Optionally, the retinyl acetate is isolated and/or further purified from the fermentation medium. Such process might comprise further steps, such as e.g. enzymatic conversion of beta-carotene into retinal via action of suitable BCOs, preferably BCOs with a selectivity towards formation of trans-retinal, more preferably leading to at least about 65-90% trans-isoforms based on the total amount of retinoids produced by said host cell, such as e.g. exemplified in WO2019/057999 and/or enzymatic conversion of retinal, particularly with a percentage of at least about 65-90% trans-retinal based on the total amount of retinoids produced by such host cell, via action of suitable retinol dehydrogenases (RDHs), as e.g. exemplified in WO2019/057998. Thus, a preferred process for production of retinyl acetate using a modified host cell as defined herein comprises the steps of (1) enzymatic conversion of beta-carotene into retinal via action of suitable BCOs, (2) enzymatic conversion of retinal into retinol via action of suitable RDHs, (3) enzymatic conversion of retinol into retinyl acetate, and optionally (4) isolation and/or purification of retinyl acetate from the fermentation medium.

The retinol and/or retinyl acetate as obtained via a process disclosed herein might be further processed/converted into vitamin A under conditions known in the art. Thus, the present invention is directed to a process for fermentative production of vitamin A using a modified host cell as defined herein.

Thus, in a particular embodiment, the present invention is directed to a process for production of a product selected from the group consisting of retinol, retinyl acetate, vitamin A, and a mix comprising retinol, retinyl acetate and vitamin A, wherein said mix comprises at least about 30% retinyl acetate based on total retinoids, said process comprising the steps of:

(a) providing a retinoid-producing host cell capable of formation of retinyl acetate,

(b) introduction of one or more modification(s) into the genome of said host cell, such as modification(s) into enzyme(s) belonging to the EC class 3.1.1.-having lipase/esterase activity, such as e.g. reducing/abolishing the enzyme activity including but not limited to deletion of the respective genes, particularly abolishment of lipase activity corresponding to Yarrowia LIP8 and optionally further abolishing enzyme activity corresponding to Yarrowia LIP2 and/or LIP3 and/or LIP4 and/or TGL1 and/or LIP16 and/or LIP17 and/or LIP18, wherein the modified host cell is still able to grow on triglyceride oils, such as e.g. vegetable corn oil, as carbon source;

(c) optionally introduction of further modification(s) comprising expression of one or more copies of (heterologous) enzymes involved in retinol, retinyl acetate and/or vitamin A production as known to a person skilled in the art,

(d) cultivation of such modified host cell under suitable conditions resulting in formation of retinol, retinyl acetate and/or vitamin A, wherein the modified host cell is grown on vegetable oil as carbon source; and

(e) optionally isolation and/or further purification of retinol, retinyl acetate and/or vitamin A from the cultivation (fermentation) medium.

A product such as retinol, retinyl acetate and/or vitamin A obtained via such process might be further used in formulations for food, feed or pharma applications as used in the art.

The modified host cell as defined herein may be cultured in an aqueous medium supplemented with appropriate nutrients under aerobic or anaerobic conditions and as known by the skilled person for the different host cells, including the presence of triglyceride oils, such as e.g. vegetable corn oil, as carbon source. The cultivation/growth of the host cell may be conducted in batch, fed-batch, semi-continuous or continuous mode. Depending on the host cell, preferably, production of retinoids such as e.g. vitamin A and precursors such as retinal, retinol, retinyl acetate can vary, as it is known to the skilled person. Cultivation and isolation of beta-carotene and retinoid-producing host cells selected from Yarrowia is described in e.g. WO2008/042338.

Carbon sources to be used for the present invention are all suitable triglyceride oils including but not limited to prehydrolysed oils containing free fatty acids like oleic, palmitic, steric or linoleic acid and glycerol, such as e.g. vegetable oil, including but not limited to corn oil, canola, safflower, sunflower, corn, soybean, or peanut oil, preferably corn oil.

“Retinoids” or a “retinoid-mix” as used herein include vitamin A, precursors and/or intermediates of vitamin A such as beta-carotene cleavage products also known as apocarotenoids, including but not limited to retinal, retinoic acid, retinol, retinoic methoxide, retinyl acetate, retinyl fatty esters, 4-keto-retinoids, 3 hydroxy-retinoids or combinations thereof. Biosynthesis of retinoids is described in e.g. WO2008/042338. A host cell capable of production of retinoids in e.g. a fermentation process is known as “retinoid-producing host cell”. The genes of the vitamin A pathway and methods to generate retinoid-producing host cells are known in the art (see e.g. WO2019/058000), including but not limited to beta-carotene oxidases, retinol dehydrogenases and/or acetyl transferases. Suitable acetyl transferase enzymes (ATFs) capable of acetylation of retinol into retinyl acetate are disclosed in e.g. WO2019/058001. Suitable beta-carotene oxidases leading to high percentage of trans-retinal are described in e.g. WO2019/057999. A “retinyl-acetate producing host cell” as used herein is expressing suitable ATFs catalyzing the conversion of retinol into retinyl acetate.

“Retinyl fatty esters” as used herein also includes long chain retinyl esters. These long chain retinyl esters define hydrocarbon esters that consists of at least about 8, such as e.g. 9, 10, 12, 13, 15 or 20 carbon atoms and up to about 26, such as e.g. 25, 22, 21 or less carbon atoms, with preferably up to about 6 unsaturated bonds, such as e.g. 0, 1, 2, 4, 5, 6 unsaturated bonds. Long chain retinyl esters include but are not limited to linoleic acid, oleic acid, or palmitic acid.

“Vitamin A” as used herein may be any chemical form of vitamin A found in aqueous solutions, in solids and formulations, and includes retinol, retinyl acetate and retinyl esters. It also includes retinoic acid, such as for instance undissociated, in its free acid form or dissociated as an anion.

“Retinal” as used herein is known under IUPAC name (2E,4E,6E,8E)-3,7-Dimethyl-9-(2,6,6-trimethylcyclohexen-1-yl)nona-2,4,6,8-tetraenal. It includes both cis- and trans-isoforms, such as e.g. 11-cis retinal, 13-cis retinal, trans-retinal and all-trans retinal. For the purpose of the present invention, the formation of trans-retinal is preferred, which might be generated via the use of stereoselective beta-carotene oxidases, such as described in e.g. WO2019/057999.

“Carotenoids” as used herein include long, 40 carbon conjugated isoprenoid polyenes that are formed in nature by the ligation of two 20 carbon geranylgeranyl pyrophosphate molecules. These include but are not limited to phytoene, lycopene, and carotene, such as e.g. beta-carotene, which can be oxidized on the 4-keto position or 3-hydroxy position to yield canthaxanthin, zeaxanthin, or astaxanthin. Biosynthesis of carotenoids is described in e.g. WO2006/102342. Cells capable of carotenoid production via one or more enzymatic conversion steps leading to carotenoids, particularly to beta-carotene, i.e. wherein the respective polypeptides involved in production of carotenoids are expressed and active in vivo are referred to herein as carotenoid-producing host cells. The genes and methods to generate carotenoid-producing cells are known in the art, see e.g. WO2006/102342. Depending on the carotenoid to be produced, different genes might be involved.

Conversion according to the present invention is defined as specific enzymatic activity, i.e. catalytic activity of enzymes described herein, including but not limited to the enzymatic activity of lipases or esterases, in particular endogenous enzymes belonging to the EC class 3.1.1.- involved in conversion of retinol into retinyl fatty esters, beta-carotene oxidases (BCOs), retinol dehydrogenases (RDHs), acetyl transferases (ATFs).

With regards to the present invention, it is understood that organisms, such as e.g. microorganisms, fungi, algae, or plants also include synonyms or basonyms of such species having the same physiological properties, as defined by the International Code of Nomenclature of Prokaryotes or the International Code of Nomenclature for algae, fungi, and plants (Melbourne Code). Thus, for example, strain Lachancea mirantina is a synonym of strain Zygosaccharomyces sp. IFO 11066, originated from Japan.

The following examples are illustrative only and are not intended to limit the scope of the invention in any way. The contents of all references, patent applications, patents, and published patent applications, cited throughout this application are hereby incorporated by reference, in particular WO2019/058000, WO2019/058001, WO2008/042338, WO2019/057999, WO2006/102342, or WO2019/057998.

### EXAMPLES

### Example 1: General Methods and Strains

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998). All genetic manipulations exemplified were performed in Yarrowia lipolytica.

Shake plate assay. Typically, 800 μl of 0.075% Yeast extract, 0.25% peptone (0.25×YP) is inoculated with 10 μl of freshly grown Yarrowia and overlaid with 200 μl of Drakeol 5 (Penreco, Karns City, Pa., USA) mineral oil with either 2% oleic acid as a carbon source in mineral. Clonal isolates of transformants were grown in 24 well plates (Multitron, 30° C., 800 RPM) in YPD media with 20% mineral oil for 4 days. The mineral oil fraction was removed from the shake plate wells and analyzed by HPLC on a normal phase column, with a photo-diode array detector.

DNA transformation. Strains are transformed by overnight growth on YPD plate media 50 μl of cells is scraped from a plate and transformed by incubation in 500 μl with 1 μg transforming DNA, typically linear DNA for integrative transformation, 40% PEG 3550 MW, 100 mM lithium acetate, 50 mM Dithiothreitol, 5 mM Tris-Cl pH 8.0, 0.5 mM EDTA for 60 minutes at 40° C. and plated directly to selective media or in the case of dominant antibiotic marker selection the cells are out grown on YPD liquid media for 4 hours at 30° C. before plating on the selective media.

DNA molecular biology. Genes were synthesized with NheI and MluI ends in pUC57 vector. Typically, the genes were subcloned to the MB5082 ‘URA3’ vector (SEQ ID NO:35) for marker selection in Yarrowia lipolytica transformations. For clean gene insertion by random nonhomologous end joining of the gene and marker HindIII/XbaI (MB5082) the restriction fragment was purified by gel electrophoresis and Qiagen gel purification column. To generate a retinyl acetate producing strain from a beta-carotene producing strain, the strain was transformed with plasmid MB9232, see Table 2, cut with SfiI and double selected for HOM3 and URA3 autotrophy. Plasmids MB9287 and MB9953, containing a Cas9, and guide RNA expression systems to target LIP2, LIP3, and LIP8 in the case of MB9287, and LIP4 in the case of MB9953, were synthesized at Genscript (Piscataway, N.J., USA).

Plasmid list. Plasmid, strains, nucleotide and amino acid sequences to be used are listed in Table 1, 2, 3 and the sequence listing. In general, all non-modified sequences referred to herein are the same as the accession sequence in the database for reference strain CLIB122 (Dujon B, et al, Nature. 2004 Jul. 1; 430(6995):35-44).

TABLE 1
list of plasmids used for construction of the strains
for overexpression or deletion of the respective genes
indicated as “Insert” or for construction used
for CRISPR/Cas9 method using the insert as gRNA driver
together with the marker as indicated. “LmATF1-mut”
refers to Lachancea mirantina (LmATF1; SEQ ID NO: 13 in
W02019058001) carrying aa substitutions
S480Q_G409A_V407I_H69A_I484L. For more details, see text.

| Plasmid | Insert | Marker |
|---|---|---|
| MB8388 | Hh/hdv, snr52 | Hyg |
| MB7452 | None (pre-Cas9) | Nat |
| MB8845 | Cas9; lip2 targeting guide RNA | Hyg |
| MB8699 | Cas9; lip3 targeting guide RNA | Hyg |
| MB9953 | Cas9; lip4 targeting guide RNA | Hyg |
| MB9373 | Cas9; lip8 targeting guide RNA | Hyg |
| MB9148 | Cas9; lip16 targeting guide RNA | Hyg |
| MB9149 | Cas9; lip17 targeting guide RNA | Hyg |
| MB9276 | Cas9; lip18 targeting guide RNA | Hyg |
| MB9702 | Cas9; tgl1 targeting guide RNA | Hyg |
| MB9282 | Cas9; Ku targeting guide RNA | Hyg |
| MB9150 | Cas9; ura3 targeting guide RNA | Hyg |
| MB9232 | LmATF1-mut | HOM3 URA3 |

TABLE 2
list of Yarrowia strains used. Construction of ML7788
and ML15710 is described in WO2016172282 (Table 2
and Ex. 5). For more details, see text or Table 1.

| Strain | Description |
|---|---|
| ML17544 | ML15710 cured of URA3 by FOA and HygR by Cre/lox |
| ML17968 | ML17544 transformed with MB8457 UmCCO1 |
| ML18183 | ML17968 transformed with MB7452 [Cas9 NatR CEN] |
| ML18210 | ML18183 transformed with MB8549 Cas9 hom3 |
| ML18210-1 | ML18210 transformed with MB9232 |
| | HOM3::LmATF1-mut::URA3 |
| ML18210-2 | ML18210-1 transformed with MB7452 [precas9] MB9282 |
| | ku70 MB9373 lip8 |
| ML18210-3 | ML18210-2 transformed with MB7452 [precas9] MB9282 |
| | ku70 MB9373 lip8 MB8845 lip2 |
| ML18210-4 | ML18210-3 transformed with MB7452 [precas9] MB9282 |
| | ku70 MB9373 lip8 MB8845 lip2 MB8699 lip3 |
| ML18210-5 | ML18210-3 transformed with MB7452 [precas9] MB9282 |
| | ku70 MB9373 lip8 MB8845 lip2 MB8699 lip3 MB9953 |
| | lip4 |

TABLE 3A
list of sequences used for construction of the plasmids/strains.
For details of the sequences, see sequence listings.

| | SEQ ID NO: |
|---|---|
| Name | (aa/nt) |
| lip2 | 1/2 |
| lip3 | 3/4 |
| lip8 | 5/6 |
| tgl-1 | 7/8 |
| lip16 | 9/10 |
| lip17 | 11/12 |
| lip18 | 13/14 |
| lip4 | 15/16 |
| est1 | 17/18 |
| lip11 | 19/20 |
| lip12 | 21/22 |
| lip20 | 23/24 |
| lip1 | 25/26 |
| lip15 | 27/28 |
| lipR | 29/30 |
| ipf3594 | 31/32 |

TABLE 3B
list of primers for CRISPR Cas9 method, PCR,
sequencing as described in Ex. 3. For more details
on the sequences, see sequence listings.

| Primer | Description | SEQ ID NO: |
|---|---|---|
| 13304 | Ku70-d-Top-66 | 36 |
| 13305 | Ku70-d-Bot-66 | 37 |
| 13308 | Ku70-c-Top-24 | 38 |
| 13309 | Ku70-c-Bot-24 | 39 |
| 12491 | ura3-Cas9-Top-66 | 40 |
| 12492 | ura3-Cas9-Bot-66 | 41 |
| 12493 | ura3-2-Top-24 | 42 |
| 12494 | ura3-2-Bot-24 | 43 |
| 14054 | lip16_pcr_rev_full | 44 |
| 14053 | lip16_pcr_for_full | 45 |
| 14052 | Lip16Dbtm | 46 |
| 14051 | Lip16Dtop | 47 |
| 13418 | LIP17-24-Bot | 48 |
| 13417 | LIP17-24-Top | 49 |
| 13324 | LIP18 rev seq | 50 |
| 13323 | LIP18 for seq | 51 |
| 13322 | LIP18 rev pcr | 52 |
| 13321 | LIP18 for pcr | 53 |
| 13315 | LIP3-Cas9-24-b-Bot | 54 |
| 13314 | LIP3-Cas9-24-b-Top | 55 |
| 13313 | LIP8-Cas9-34-Bot | 56 |
| 13312 | LIP8-Cas9-34-Top | 57 |
| 13259 | LIP18-Cas9-24-Bot | 58 |
| 13258 | LIP18-Cas9-24-Top | 59 |
| 13257 | LIP18-Cas9-66-Bot | 60 |
| 13256 | LIP18-Cas9-66-Top | 61 |
| 13147 | LIP17 rev seq | 62 |
| 13146 | LIP17 for seq | 63 |
| 13145 | LIP17 rev pcr | 64 |
| 13144 | LIP17 for pcr | 65 |
| 13143 | LIP16 rev seq | 66 |
| 13142 | LIP16 for seq | 67 |
| 13141 | LIP16 rev pcr | 68 |
| 13140 | LIP16 for pcr | 69 |
| 13111 | LIP17-Cas9-24-Bot | 70 |
| 13110 | LIP17-Cas9-24-Top | 71 |
| 13109 | LIP17-Cas9-66-Bot | 72 |
| 13108 | LIP17-Cas9-66-Top | 73 |
| 13107 | LIP16-Cas9-24-Bot | 74 |
| 13106 | LIP16-Cas9-24-Top | 75 |
| 13105 | LIP16-Cas9-66-Bot | 76 |
| 13104 | LIP16-Cas9-66-Top | 77 |
| 12850 | Lip8 rev seq | 78 |
| 12849 | Lip8 for seq | 79 |
| 12848 | Lip8 rev pcr | 80 |
| 12847 | Lip8 for pcr | 81 |
| 12840 | LIP2ioRevXba | 82 |
| 12839 | LIP2ioFwdMlu | 83 |
| 12838 | LIP2iorevMlu | 84 |
| 12837 | LIP2ioFwdkpn | 85 |
| 12821 | LIP8-Cas9-24-Bot | 86 |
| 12820 | LIP8-Cas9-24-Top | 87 |
| 12819 | LIP8-Cas9-66-Bot | 88 |
| 12818 | LIP8-Cas9-66-Top | 89 |
| 12707 | LIP2 for seq | 90 |
| 12706 | LIP2 rev pcr | 91 |
| 12705 | LIP2 for pcr | 92 |
| 12602 | LIP2-Cas9-24-Bot | 93 |
| 12601 | LIP2-Cas9-24-Top | 94 |
| 12600 | LIP2-Cas9-66-Bot | 95 |
| 12599 | LIP2-Cas9-66-Top | 96 |
| 12564 | LIP3 rev seq | 97 |
| 12563 | LIP3 for seq - (really reverse) | 98 |
| 12562 | LIP3 rev pcr | 99 |
| 12561 | LIP3 for pcr | 100 |
| 12464 | LIP3-Cas9-24-Bot | 101 |
| 12463 | LIP3-Cas9-24-Top | 102 |
| 12462 | LIP3-Cas9-66-Bot | 103 |
| 12461 | LIP3-Cas9-66-Top | 104 |
| 14025 | tglseq-rev | 105 |
| 14024 | tglseq-fwd | 106 |
| 14023 | TglDelta-rev | 107 |
| 14022 | TglDelta-fwd | 108 |
| 13307 | Ku70-e-Bot-66 | 109 |
| 13306 | Ku70-e-Top-66 | 110 |
| 12074 | ku70RightseqFwd | 111 |
| 12073 | ku70LeftseqFwd | 112 |
| 14152-2 | Lip4-5′-top-24 | 113 |
| 14152-3 | Lip4-5′-bot-24 | 114 |
| 14152-4 | Lip4-3′-top-66 | 115 |
| 14152-5 | Lip4-3′-bot-66 | 116 |
| 14151 | LIP4-5′seq-fwd | 117 |
| 14152 | LIP4-3′seq-rev | 118 |

Fermentation conditions. Fermentations were identical to the previously described conditions using mineral oil overlay and stirred tank in a bench top reactor with 0.5 L to 5 L total volume (see WO2016/172282, Ex. 5 and 6 but with a different oil), however, they were oleic acid fed. Generally, the same results were observed with a fed batch stirred tank reactor with an increased productivity, which demonstrated the utility of the system for the production of retinoids. Preferably, fermentations were batched with 6% glucose and 20% mineral oil was added after dissolved oxygen dropped below about 20% and feed was resumed to achieve 20% dissolved oxygen throughout the feeding program. Fermenters were harvested and compared at 138 hrs.

UPLC reverse phase retinol method. For rapid screening this method does not separate cis-isomers, only major functional groups. A Waters Acquity UPLC with PDA detection (or similar) with auto sampler was used to inject samples. An Acquity UPLC HSS T3 1.8 um P/N 186003539 was used to resolve retinoids. The mobile phase consisted of either, 1000 mL hexane, 30 mL isopropanol, and 0.1 mL acetic acid for retinoid related compounds. Column temperature was 20° C. The injection volume was 5 μL. The detector was a photodiode array detector collecting from 210 to 600 nm. Analytes were detected according to Table 4.

TABLE 4A
list of analytes using reverse phase retinol method. The addition
of all added intermediates gives the total amount retinoids. Beta-
carotene* can be detected in 325 nm and will interfere with retinyl
ester quantitation, therefore care must be taken to observe the
carotene peak and not include them in the retinoid quantification.
“N/A” means “not available”. For more details, see text.

| | Retention | | |
|---|---|---|---|
| | time | Lambda max | Response |
| Intermediates | [min] | [nm] | factor |
| retinyl-acetate | 2.93 | 325 | 1.00 |
| retinyl-esters | 3.2-3.8 | 325 | 1.68 |
| retinal | 2.77 | 325 | 0.87 |
| retinol | 2.73 | 325 | 0.87 |
| Beta-carotene* | 3.56 | 450 | N/A |

TABLE 4B
UPLC Method Gradient with solvent A: water; solvent B: acetonitrile;
solvent C: methanol; solvent D: tert-butyl methyl ether.

| Time | | | | | Flow | Pressure |
|---|---|---|---|---|---|---|
| [min] | % A | % B | % C | % D | [ml/min] | [psi/bar] |
| 0 | 50 | 50 | 0 | 0 | 0.5 | 9500-14000max |
| 0.5 | 50 | 50 | 0 | 0 | 0.5 | |
| 1.0 | 0 | 50 | 50 | 0 | 0.5 | |
| 1.25 | 0 | 0 | 100 | 0 | 0.5 | |
| 3.25 | 0 | 0 | 5 | 95 | 0.5 | |
| 3.5 | 0 | 0 | 5 | 95 | 0.5 | |
| 4.0 | 0 | 0 | 100 | 0 | 0.5 | |
| 4.25 | 0 | 50 | 50 | 0 | 0.5 | |
| 4.5 | 50 | 50 | 0 | 0 | 0.5 | |

Method Calibration. Method is calibrated on retinyl acetate, retinols and retinals are quantitated against retinyl-acetate using the indicated response factor. Retinyl Acetate is dissolved in THF at −200 μg/ml for stock solution using a volumetric flask. Using volumetric flasks, ×20, ×50 and ×100 dilutions of stock solution in 50/50 methanol/MTBE were made. UV absorbance of retinyl acetate becomes nonlinear fairly quickly, so care must be taken to stay within the linear range. Consequently, lower concentrations might be better. Retinyl palmitate can also be used as retinyl ester calibration.

Sample preparation. Samples were prepared by various methods depending on the conditions. For whole broth or washed broth samples the broth was placed in a Precellys® tube, weighed, and mobile phase was added. Briefly in a 2 ml Precellys® tube, add 25 μl of well mixed broth and 975 μl of THF. The samples were then processed in a Precellys® homogenizer (Bertin Corp, Rockville, Md., USA) on the highest setting 3× according to the manufacturer's directions, typically 3 repetitions×15 minutes×7500 rpms. For the washed pellet the samples were spun in a 1.7 ml tube in a microfuge at 10000 rpm for 1 minute, the broth decanted, 1 ml water added, mixed, pelleted and decanted, and brought up to the original volume. The mixture was pelleted again and brought up in appropriate amount of mobile phase and processed by Precellys® bead beating. For analysis of mineral oil fraction, the sample was spun at 4000 RPM for 10 minutes and the oil was decanted off the top by positive displacement pipet (Eppendorf, Hauppauge, N.Y., USA) and diluted into mobile phase mixed by vortexing and measured for retinoid concentration by UPLC analysis.

### Example 2: Lipase/Esterase Overexpression in Yarrowia lipolytica

To test the influence of endogenous lipases and/or esterases on production of retinoids in a suitable Yarrowia host, overexpression experiments were carried out, wherein only 1 gene at the time was overexpressed (no combination of 2 or more genes).

Lipases were overexpressed as described above (Example 1). Native Yarrowia lipase genes were synthesized and sequence verified by GenScript then cloned into the NheI and MluI sites of MB5082. The genes are TEF1 promoter driven that allows selection for by complementation of an uracil auxotroph strain (ura3).

Plasmids containing the respective lipase/esterase genes cleaved by XbaI/HindIII were transformed into retinoid producing strain ML18210-9 carrying the wild-type lip8 gene (see Example 1, Table 2) and selected for uracil prototrophy. Clonal isolates of transformations were grown for four days in 0.25× Yeast/Peptone (YP) with 2% corn oil as a carbon source and a 20% mineral oil overlay in the standard shake plate assay and assayed by the previously described UPLC analytical method. At least two individual clonal isolates of transformed Yarrowia strains were tested by shake plate and measured by UPLC assay % retinyl esters and % retinol per mass of total retinoids. The result is depicted in Table 5, showing production of retinyl fatty esters and retinol. Best performance on accumulation of retinyl fatty esters and conversion of retinol is achieved with overexpression of LIP8, some minor effect was visible with LI P3 overexpression.

TABLE 5
performance of Yarrowia strains overexpressing single endogenous
lipases or esterases as indicated. The percentage of retinyl
esters (“% esters”) and retinol (“% retinol”)
based on the total amount of retinoids is given. Empty vector
is the plasmid without an ORF inserted, that can be interpreted
as a negative control. For more details, see text.

| Insert | % esters | % retinol |
|---|---|---|
| empty | 8 | 26 |
| LIP3 | 24 | 18 |
| LIP8 | 95 | 3 |
| TGL1 | 8 | 43 |
| LIP16 | 7 | 61 |
| LIP17 | 8 | 65 |
| LIP18 | 9 | 45 |
| EST1 | 7 | 25 |
| LIP11 | 6 | 26 |
| LIP12 | 6 | 25 |
| LIP20 | 7 | 25 |
| LIP1 | 6 | 26 |
| LIP15 | 7 | 25 |
| LIPR | 7 | 24 |
| IPF3594 | 5 | 25 |

### Example 3: Deletion of Lipase Genes in Yarrowia lipolytica

Lipase genes were deleted using modern CRISPR Cas9 methods. The strains were pre-transformed with MB7452 expressing Cas9 (SEQ ID NO:34) under nourseothricin selection, that increased the deletion frequency when a subsequent guide RNA was transformed. Cas9 guide RNAs were selected using the Geneious® 10.1.3 software (Biomatters Ltd). Sites were selected that are as close to the beginning of the open reading frame (ORF) for single cuts or at 5′ and 3′ to remove most of gene. Guides were inserted into SapI cloning sites of the vector MB8388 (SEQ ID NO:33) and were synthesized and sequence verified by GenScript (see Table 3 for sequences). Strains were transformed and selected on YPD Hygromycin at 200 μg/ml then replica plated to YPD. Plasmids are passed by outgrowth on YPD plates containing Nourseothricin 100 μg/ml and replica plating to YPD Hygromycin at 200 μg/ml to identify colonies that have lost the guide RNA fragment, but still contain the PreCas9 plasmid, MB7452. Then these clones were screened for deletion by PCR over the gene using primers 100 bp upstream and 100 bp downstream, identifying the deletion by gel mobility, and sequencing the deletion. To precisely remove the ORF for the Cas9 deletions template DNA (100 bp with 50 bp 5′ of the ORF, and 50 bp 3′ of the ORF as in strain CLIB122) was used in strains where the ku70 gene (YALI0008701g) was previously deleted using MB9282. Sequences of the guide RNA expressing region are referenced in Table 3. Nucleotides that code for guides in the sequence anneal and ligate to the SapI sites and result in removal of the SapI site that was present in the oligonucleotide. The annealing of the guides is directed by the specific overhangs in the guide sequence (5′ to 3′ on the top strand: ATG, GTT, CGT, TTT). The first three nucleotides of the guide containing the SapI site is included in the insert sequence for clarity in alignment and the annealed overhangs can be assembled into the vector MB8388 (SEQ ID NO:33) by matching the overhangs. The 24 base pair inserts are inserted into a guide RNA that is driven and processed by a hammerhead ribozyme system (hh, hdv), and the 66 base pair insert is driven by the Yarrowia SNR52 promoter. Single stranded oligonucleotides can assemble the guide sequences by annealing top and bottom sets and using these for ligation into appropriate the SapI sites. Plasmids containing these inserts in MB8388 have been routinely synthesized at the DNA provider GenScript, (Piscataway, N.J., USA). Examples of the oligonucleotides used in these assemblies are included in Table 3B.

### Example 4: Effect of Lipase Knockouts on Formation of Retinyl Acetate

To explore the effects-on retinyl acetate production, we constructed lipase deletions in retinyl acetate producing strain ML18210-1 expressing a highly active acetyl transferase derived from Lachancea mirantina, i.e. LmATF1 (see WO2019058001: SEQ ID NO:13), carrying amino acid substitutions S480Q_G409A V407I_H69A_I484L. The lineage of said strain is known from Table 2. Removal of the open reading frames of lipase genes was carried out using CRISPR Cas9 methods. This scheme was performed by primary introduction of a ku70 mutation, using MB9282 and subsequently co-transforming lipase deletion plasmids with template DNA (100 nucleotide base pairs 5′ and 3′ of the ORF ordered as FragmentGENE from Genewiz.com, Cambridge, Mass., USA) that directs a precise deletion of the ORF, since homologous recombination is required to repair the double strand break in a ku70 mutant. Deletion of only one or several lipase genes, i.e. serial deletion, was performed with this technique. Said modified strains were tested for formation of retinoids, in particular formation of retinyl acetate, as shown in Table 6, with focus on purity, i.e. the percentage of retinyl acetate based on the total amount of retinoids, and abundance, i.e. comparison between retinyl acetate formation with a lipase-deleted strain to retinyl acetate formation with strain ML18210-1 (wild-type strain for all endogenous lipase genes). Strains were grown in 2% oleic acid in 0.25× yeast peptone fed shake plate and fermentations with a 20% mineral oil overlay for four days at 30° C. in shake plates as described in Example 1. The results are shown in Table 6 for deletion of LIP8 alone, leading to a percentage of 70% retinyl acetate based on total retinoids, or in combination with LIP2 and/or LIP8 and/or LIP4, with some further increase of the percentage. Addition of further deletions selected from TGL1 and/or LIP16 and/or LIP17 and/or LIP18 might result in at least the same retinyl acetyl percentages, i.e. in the range of at least about 70-90% retinyl acetate based on total retinoids, with further increase of at least about 10% compared to retinyl acetate formation with deletion of LIP8 only.

TABLE 6
Effect of lipase deletions on purity and abundance of retinyl acetate
formation in a retinyl acetate-producing Yarrowia host. “% retAc”
means purity of retinyl acetate, “increase [%]” means abundance
of retinyl acetate with the value for strain ML18210-2 being zero,
“deletion” refers to the deleted genes. For further details, see text.

| ML strain | deletion | % retAc | increase [%] |
|---|---|---|---|
| 18210-1 | N/A | N/A | N/A |
| 18210-2 | lip8 | 70% | 0 |
| 18210-3 | lip8 lip2 | 78% | 12 |
| 18210-4 | lip8 lip2 lip3 | 92% | 31 |
| 18210-5 | lip8 lip2 lip3 lip4 | 94% | 32 |

1. A retinoid-producing host cell capable of retinyl acetate formation, particularly retinyl acetate-producing host cell, such as fungal host cells, preferably oleaginous yeast cell such as e.g. Yarrowia, comprising one or more genetic modification(s), such as reduction or abolishment, preferably abolishment, of endogenous enzymes involved in pre-digestion of vegetable oil into glycerol and fatty acids, preferably endogenous enzymes belonging to EC class 3.1.1.-, more preferably enzymes with esterase or lipase activity.

2. The host cell according to claim 1, wherein the expression of endogenous genes is reduced or abolished, preferably abolished, said genes encoding enzymes with activities corresponding to enzyme activities selected from the group consisting of Yarrowia LIP2, LIP3, LIP4, LIP8, TGL1, LIP16, LIP17, LIP18, and combinations thereof.

3. The host cell according to claim 1, wherein the modification leads to an increase in the percentage of retinyl acetate to at least about 70%, such as at least about 70-90%, based on total retinoids compared to a host cell, wherein the respective genes are still expressed and active.

4. The host cell according to claim 1, comprising a modification in a polypeptide obtainable from Yarrowia lipolytica with at least about 50%, such as 60, 70, 80, 90, 95, 98, or 100% identity to a polypeptide selected from the group consisting of SEQ ID NO:1, 3, 5, 7, 9, 11, 13, 15 and combinations thereof.

5. The host cell according to claim 1, wherein the endogenous enzyme corresponding to Yarrowia LIP8 is reduced or abolished, preferably abolished.

6. The host cell according to claim 1, wherein formation of retinal acetate is increased during fermentation compared to the formation of retinyl acetate using the respective non-modified host cell, and wherein a percentage of at least about 70%, such as e.g. about 75, 80, 85, 90, 95, 98% or more, including 100%, retinyl acetate based on total retinoids present in/produced by said modified host cell is obtained.

7. The host cell according to claim 1 used in a fermentation process for production of retinoids with vegetable oil as carbon source, wherein the percentage of retinyl acetate present in said retinoid mix is about 70% or more, preferably about 75, 80, 85, 90, 95, 98% or more, including 100%, retinyl acetate based on total retinoids present in or produced by said host cell.

8. The host cell according to claim 1, wherein the host cell is selected from Yarrowia, preferably Yarrowia lipolytica, comprising inactivation, preferably deletion, of the LIP8 gene, optionally combined with inactivation, preferably deletion, of a gene selected from the group consisting of LIP2, LIP3, LIP4, TGL, LIP16, LIP17, LIP18, and combinations thereof.

9. Use of a host cell according to claim 1 in a process for production of retinoids selected from the group consisting of retinol, retinyl acetate, retinyl fatty esters, vitamin A or mixtures thereof.

10. Use according to claim 9, wherein the percentage of retinyl acetate is in the range of about 70% or more based on the total amounts of retinoids.

11. Use according to claim 9, wherein the host cell is grown on vegetable oil as carbon source, preferably corn oil.

12. A process for reducing or abolishing the percentage of retinoids other than retinyl acetate in a retinoid mix generated in a fermentation process, comprising the steps of:
(1) introducing into a retinoid-producing host cell heterologous genes encoding enzymes involved in retinol to retinyl acetate conversion and optionally enzymes involved in retinal to retinol conversion and/or beta-carotene to retinal conversion,
(2) introducing one or more modification(s) in endogenous enzyme activities involved in pre-digestion of vegetable oil into glycerol and fatty acids, preferably enzymes, belonging to EC class 3.1.1.-, more preferably enzymes having lipase or esterase activity, most preferably with activities corresponding to Yarrowia LIP8, LIP2, LIP3, LIP4, TGL, LIP16, LIP17, LIP18, and combinations thereof, wherein the modification is a reduction or abolishment of such enzyme activity, preferably abolishment of said enzyme activity.

13. A process for production of a product selected from the group consisting of retinol, retinyl acetate, vitamin A, and a mix comprising retinol, retinyl acetate and vitamin A, said process comprising the steps of:
(a) providing a retinoid-producing host cell capable of formation of retinyl acetate,
(b) introduction of one or more modification(s) into the genome of said host cell, such as modification(s) into enzyme(s) belonging to the EC class 3.1.1.- having lipase activity, such as e.g. reducing/abolishing the enzyme activity including but not limited to deletion of the respective genes, particularly abolishment of lipase activity corresponding to Yarrowia LIP8 and optionally further abolishing enzyme activity corresponding to Yarrowia LIP2 and/or LIP3 and/or LIP4 and/or TGL and/or LIP16 and/or LIP17 and/or LIP18, wherein the modified host cell is still able to grow on vegetable oil as carbon source;
(c) optionally introduction of further modification(s) comprising expression of one or more copies of (heterologous) enzymes involved in retinol, retinyl acetate and/or vitamin A production as known to a person skilled in the art,
(d) cultivation of such modified host cell under suitable conditions resulting in formation of retinol, retinyl acetate and/or vitamin A, wherein the modified host cell is grown on vegetable oil as carbon source; and
(e) optionally isolation and/or further purification of retinol, retinyl acetate and/or vitamin A from the cultivation (fermentation) medium.

14. A process for the identification of suitable endogenous hydrolases to be modified in order to increase the percentage of retinyl acetate in a fermentation of a retinyl acetate producing host cell grown on vegetable oil as carbon source, comprising the steps of: pre-digestion of vegetable oil into glycerol and fatty acids,
(2) selection of endogenous lipase or esterase enzymes based on sequence homology of at least about 50%, such as e.g. 60, 70, 80, 90, 95, 98 or 100% to SEQ ID NO:1, 3, 5, 7, 9, 11, 13, 15,
(3) overexpression of selected genes and comparison of retinyl acetate percentage based on total retinoids,
(4) selection of genes, wherein overexpression had a negative impact on retinyl acetate percentage in the retinoid mix, and
(5) reduction or abolishment, e.g. inactivation, such as e.g. via deletion, of selected genes for enhancement of retinyl acid formation in a retinoid mix.


SEQUENCE LISTING (118 sequences)

SEQ ID NO: 1 - protein, 334 aa, Yarrowia lipolytica
MKLSTILFTACATLAAALPSPITPSEAAVLQKRVYTSTETSHIDQESYNFFEKYARLANIGYCVGPGTKIFKPFNCGLQCAHFPNVELIEEFHDPRLIFDVSGYLAVDHASKQIYLVIRGTHSLEDVITDIRIMQAPLTNFDLAANISSTATCDDCLVHNGFIQSYNNTYNQIGPKLDSVIEQYPDYQIAVTGHSLGGAAALLFGINLKVNGHDPLVVTLGQPIVGNAGFANWVDKLFFGQENPDVSKVSKDRKLYRITHRGDIVPQVPFWDGYQHCSGEVFIDWPLIHPPLSNVVMCQGQSNKQCSAGNTLLQQVNVIGNHLQYFVTEGVCGI

SEQ ID NO: 2 - DNA, 1005 bp, Yarrowia lipolytica
atgaagctttccaccatcctcttcacagcctgcgctaccctggctgccgccctcccttcccccatcactccttctgaggccgcagttctccagaagcgagtgtacacctctaccgagacctctcacattgaccaggagtcctacaacttctttgagaagtacgcccgactcgcaaacattggatattgtgttggtcccggcactaagatcttcaagcccttcaactgtggcctgcaatgtgcccacttccccaacgttgagctcatcgaggagttccacgacccccgtctcatctttgatgtttctggttacctcgctgttgatcatgcctccaagcagatctaccttgttattcgaggaacccactctctggaggacgtcataaccgacatccgaatcatgcaggctcctctgacgaactttgatcttgctgctaacatctcttctactgctacttgtgatgactgtcttgtccacaatggcttcatccagtcctacaacaacacctacaatcagatcggccccaagctcgactctgtgattgagcagtatcccgactaccagattgctgtcaccggtcactctctcggaggagctgcagcccttctgttcggaatcaacctcaaggttaacggccacgatcccctcgttgttactcttggtcagcccattgtcggtaacgctggctttgctaactgggtcgataaactcttctttggccaggagaaccccgatgtctccaaggtgtccaaagaccgaaagctctaccgaatcacccaccgaggagatatcgtccctcaagtgcccttctgggacggttaccagcactgctctggtgaggtctttattgactggcccctgatccaccctcctctctccaacgttgtcatgtgccagggccagagcaataaacagtgctctgccggtaacactctgctccagcaggtcaatgtgattggaaaccatctgcagtacttcgtcaccgagggtgtctgtggtatctaa

SEQ ID NO: 3 - protein, 498 aa, Yarrowia lipolytica
MPLELPSLNASIVGNTVQNGAVEQFLNIRYADIPGKFEKPVLKNDWNGAEIDATKVGPVCPQPRTPFNFFSVPDDLWEKVNVDTYQDGLLCDNLIVTRPKGVSANARLPTVVWIHGGSNIEGSIYNLIYEPQFLVAESVRVGKPIVHVCIEYRLGLAGFLTKNGKGNWGTWDQYTGCQWVNRHIQDFGGDPLNVTLTGESAGSVAVHNMLIKDSMNGRKLFRNAVMMSGTLETITPQPPKWHARLEEKVAKVTGKEVADLASLSDKELLDAQIKLNVAVCMTCDDGDFFEPGWKQHLTPDWLDKLIISDCKDEGMLYFLPVNAQDDEELLAKVAKSPVGKEISELYGIKEGGDIKSACLDLKTDATFNYFNHLLFKKMEEARNNGSTSRVYRLAVDEPNPHNPDQRAHHAVDVLYMFNSTKFNEHGDKLSRLFQSHFLRLAYGLEPWDHRNFGVYRNGGYQQLPLSELNKVRPVERYEALSKMDFGQVGRLSNALSRL

SEQ ID NO: 4 - DNA, 1497 bp, Yarrowia lipolytica
atgcctctcgaactcccctcgctcaacgcctcgattgtcggcaacaccgttcagaacggcgctgttgagcagtttctcaacatccgatacgccgacattcctggcaagtttgagaagcccgtgctcaagaacgattggaacggcgcggagatcgacgccaccaaggtcggtcccgtgtgcccccaaccccgcaccccattcaacttcttctccgtgccagacgacctctgggagaaagtcaatgtggacacgtaccaggacggtctgctgtgcgacaacctgattgtgacgcgaccgaagggcgtgtctgccaacgcccggctgcccactgttgtgtggatccacggcggctccaatattgagggcagtatctacaacctcatctatgagccccagttcctggtggcagagtcggtgcgagtaggcaagccgattgtgcacgtgtgtatcgagtaccgactgggtctcgcgggcttcctcaccaagaacggcaagggcaactggggcacgtgggatcagtacacgggctgccagtgggtcaaccgccacattcaggactttggaggcgatcctttgaacgtgacattgaccggtgagtctgccggctctgtagcagtccataacatgctcatcaaggactccatgaacggccgaaagttgttccgaaatgccgtcatgatgagtggcactctcgagaccatcactcctcagcctcccaagtggcatgctcgtttggaggagaaggtggccaaggtcactggcaaggaagtggccgaccttgcttctctgtccgataaggagctgctcgacgcccagatcaagctcaatgtggctgtgtgcatgacttgcgacgacggcgactttttcgagcccggatggaagcagcatctgactcctgactggctcgacaagctcatcatctccgattgcaaggacgagggcatgctgtatttcctgccagtcaacgcgcaggacgacgaggagctgttggcaaaggtggccaagtcgcccgtgggtaaggagatttccgagctttacggcatcaaggagggtggcgatatcaagtctgcgtgtctcgatctcaagactgacgccaccttcaattactttaaccatctgctgttcaagaagatggaggaggcccgaaacaacggctccacttctcgagtttaccgtctggccgtcgatgagcccaacccccacaaccccgaccagcgggcccaccacgccgtcgacgtgctgtacatgttcaactcgaccaagttcaacgagcacggcgacaagctgtctcggctgttccagagccactttttgcggctggcgtatggcctggagccctgggaccatcgaaactttggagtgtacagaaacggcggctaccagcagctgccgctgagtgagttgaacaaggtccgacccgtcgagcggtacgaggcgctgtccaagatggactttggccaggttgggcgtttgtccaatgcgctttcgcgcctatga

SEQ ID NO: 5 - protein, 371 aa, Yarrowia lipolytica
MVSLSARIKDFFSVLLLGAATITPSTQTAGVSQGFYDFARDFAHLSNIAYCVNAPITPLNPDFTCGNSCKHFPEIELVKTFGGNFFKTSITGYLAVDHVKKEKYVVFRGTFSLADAITDMQFQLSPFLVDVPALNTFSANDTTAEAQTHCEGCKIHDGFSKAFTETWGNIGEDLQKHLDANPDYQLYVTGHSLGAAMALLGATSIKLKGYDPILINYGQPRVGNKPFAEFINKLWFGEGNGLEITPERKLYRMTHWNDIFVGLPNWEGYTHSNGEVYINNRFINPPLKDVISCAGGENSKCYRSSFSLLSQINLLQNHLAYIDYIGYCALNIGRRELADQEHYTGPYYYGHRSEEDFKKLGLELSTPQVEN

SEQ ID NO: 6 - DNA, 1116 bp, Yarrowia lipolytica
atggtatccctctctgctcgaatcaaagactttttttcggtcctcctcctcggagctgcaaccatcactccctccacacagaccgcaggcgtgtctcaagggttctatgattttgctcgggactttgcccatctgtccaacattgcctactgtgtcaatgctcccatcactccactgaacccggacttcacctgtggcaactcgtgcaagcactttccggaaattgagcttgtgaagacatttggaggcaacttcttcaagacctccattacgggctacctggctgtcgatcatgtcaagaaggagaagtacgttgtcttccgaggaaccttttcgctggcagacgcgatcacggacatgcagttccagctgtctcctttcctggtcgatgtgcctgccctgaacactttctcagctaatgacaccaccgcagaggcccagacgcactgtgagggctgcaaaattcacgacggcttctccaaggcctttaccgagacctggggtaacattggtgaggatctgcagaaacacctggacgctaacccggactaccagctgtacgtgactggccattctctgggagctgctatggcccttcttggagctacttccatcaagctcaagggctacgatcccattctcatcaactacggacagccccgagtcggaaacaagcccttcgctgagttcattaacaagttgtggtttggagaaggcaacggtctggaaatcacccccgagagaaagctgtaccgaatgacccactggaacgacatctttgttggcctgcccaactgggagggatacacccactctaacggtgaagtatacatcaacaaccggttcatcaaccctcctctcaaggatgtcatctcttgtgctggaggcgaaaactcgaagtgctaccgatcctcgttcagcctgctgtcccagatcaatctgctccaaaaccacctggcttacattgattacattggatactgcgctctgaacattggtcgacgagagcttgccgatcaggaacattacactggtccttattactatggtcatcgatctgaggaggactttaagaagttgggcttggagctatccaccccacaagttgagaactga

SEQ ID NO: 7 - protein, 569 aa, Yarrowia lipolytica
MHVPVLGRLELSHWPMLFVSLFLVCLEFLVSFITALLPNFFIEWCHDKVEILQVYFPSTGEVALRKSPEQLETVNALLKARDFVEICDFHGYQAEEHVVQTKDGFLLGVHRILPKNPAALLTDDPEVLSTPAPPPAFLAGNLEVYSSPPKRGSKRSVSSKLITRPVVYLHHGLLMNSEVWVVNTDAKKSIAFALADLGFDVWLGNNRGNKYSRKHMKYNPESREFWDFCLDDFALFDIPDSIDYILSVTKQKSLSYIGFSQGSAQAFASLAIRPPLNDKVNLFIAVAPAMSPPGLRSKIVNSLMRASPQLLFLCFGRRAILSSSPFWESVLDAKLYARIIDAACRMLFDWYGENITWPQKIAAYHHLYSYTSVKSVVHWFQIIRAASFHMFEDVINSPLDPHLCKYSTVTRYPTENIRTPIALIYGKTDSLVDIDQMLSTLPSSTIAFGVPKHEHLDLLWGDEADTLVIPKIVALLDYYTPVPRSNKSSSDHISERLFEPANSKVSGLGLSQDLKSPLSLKKRSLSNSSSIQDSHKVGGDTDSVLSLRNLTPKGYALGSAKPVSGTLNP

SEQ ID NO: 8 - DNA, 1710 bp, Yarrowia lipolytica
atgcacgtccccgttctaggacgtctcgagctgtcacattggcccatgctgtttgtgtcgctcttcctcgtctgcctcgagtttctcgtgagcttcatcaccgctctgctccccaacttcttcattgaatggtgccacgacaaggtcgagattctgcaggtctacttccccagcaccggcgaggttgctctcagaaagtctcccgagcagctcgaaaccgtcaacgctctgctcaaggcccgagactttgttgaaatctgcgatttccatggctaccaggccgaagaacatgtggttcagaccaaggatggatttctgctaggagtgcaccgaatcctgcccaagaaccctgctgctctgctcaccgacgaccccgaagtcctcagcacccccgcccctcccccggccttcctggctggaaacctcgaagtctactcttcgccccccaaaagaggctccaagcgaagtgtgtcctccaaactgatcacccgacccgttgtctaccttcaccatggtctgctgatgaactcggaggtctgggttgtgaacacggacgccaaaaagtcgattgcttttgctctggccgacctgggatttgacgtctggctcggaaacaacagaggcaacaagtactcgcgaaaacacatgaaatacaaccccgagtcgcgcgagttctgggatttctgtctcgacgactttgcgctgtttgatatccccgattccattgactatattctgtctgtgaccaaacagaagtctctgtcttacattggattctcccagggttcagcccaggcatttgcatctctggctatccgacctcctctcaacgataaggtcaacctattcattgccgttgcccccgccatgtctcctcctggcctgagatccaagattgtcaactccctcatgagagcttctccacagctgctgttcctctgctttggtcgaagagctatcttgtcatcctctcccttctgggagtcggttctggacgcaaaactgtatgctcgaatcatcgatgctgcctgcagaatgctgtttgactggtacggagagaacatcacctggccccagaagattgcagcctaccaccacctgtactcgtacacctctgtcaagtccgtcgtgcattggttccagatcatccgagcagcctccttccacatgtttgaggatgtcatcaactctcccctggaccctcatctgtgcaagtactccactgtcacccgataccccaccgagaacattcggacacccattgctctgatctacggcaagaccgactcgctggtggacattgaccagatgctgtctactctaccctcatccactattgcatttggagtccccaaacatgagcatctcgatcttctgtggggagacgaagccgacactctggttatccccaagattgtcgctctgctcgattattacacacctgtgcccagaagtaacaagagcagcagtgaccatatcagcgagcgtctatttgagcctgctaactccaaggtctcgggtctgggactcagccaggacctcaagtcgccattgtctttgaaaaagagatctctctccaactcctcatcgatccaggactctcataaggtcggaggagacacagactcggttctaagtctccgaaacctcactcccaaggggtacgctcttggaagtgccaagccagtgagtggcactctcaacccttga

SEQ ID NO: 9 - protein, 365 aa, Yarrowia lipolytica
MLSLIAIFLLVTTALAQTANITQSTYDFVLKYGRLSNVAYCVKAPGPYELETDFTCGRSCGHFPNVTLEHQFGGDFFSTSITGFLAHDHTKKEKYIVFRGTFSLADAITDALFLQEPYLADLPPLNTTNINSTSNSARVDCPDCEIHDGFQKAYRETMVNMQGHLVAFLRNNTDYKLIVTGHSLGAATALLMGINLKNLGFDPMVITFGQPRVGNKAFADYADSLFFKQGDNGLNINPERRLYRVTHWNDIVVGVPFWSGYTHTLGEVYISYPDGVNAPIEYVNACAGPDNDQCHYGSFDLLARVNILKNHCAYLNWIFYCAFNVDKRQMMIDPPRIHKRVEHWSGKFADVEFSERMIYEATYPM

SEQ ID NO: 10 - DNA, 1243 bp, Yarrowia lipolytica
agtgagtattgactggaaagtttgcacaagttcgcaaacacaagattgagattggtataggtccaatgtccactagagccgatacgagctgtctacaattgcctgaatagctcctccatctcgtcaatcgttccgcactaacaaagtgctctcgttaattgccatctttctacttgtcaccacggccctcgctcaaacagccaacatcacgcagtcgacctacgacttcgttctcaagtacggacgactctccaatgtggcctattgtgtcaaggcccccggtccatacgaactggagacggacttcacctgtggaagatcttgcggtcactttcccaacgtgacgttggaacaccagtttggaggcgactttttctccacgtccataaccggctttctggcccacgatcacactaagaaagagaaatacattgttttccgaggcaccttttctctggcagacgccatcaccgacgccttgtttctccaggagccctacctggccgatctgccgcctctgaacaccaccaacatcaactccactagcaattcggcccgagtcgactgccccgattgtgagattcatgacggcttccaaaaggcataccgagagaccatggtgaacatgcagggccacctggtggcctttcttagaaacaacacagactacaagctgatagtcaccggccattctctgggagccgccactgctctgctcatgggcatcaacctgaagaatctcggctttgaccctatggtgatcacgtttggacagcctcgagtgggaaacaaggcgtttgcggactacgccgactcgctctttttcaaacagggagataacggactgaatatcaaccctgaaagacgtctttaccgagtcactcattggaatgacatcgtggtgggagtgcccttctggtccggttatacccatactctgggagaagtgtacatcagctacccagacggagtcaacgcccccatcgaatacgtcaatgcgtgcgctggaccagacaacgaccagtgccactacgggtccttcgacctgctggctcgagtcaacattctcaaaaaccactgtgcctatctcaactggatcttctactgcgcctttaacgtggacaagagacagatgatgattgatcctccgagaatccacaagagagtcgagcattggagcggcaagtttgcagacgtcgagttcagcgagagaatgatctacgaggccacttatcccatgtga

SEQ ID NO: 11 - protein, 363 aa, Yarrowia lipolytica
MLSFIALFLLVAQALAQTAPITQETYDFVLKYGWLSNVAYCVRAPGPFALQSDFTCGNSCAHFPDVTLDYQFGGNFFSTSVTGFLAHDHTKKEKYIVFRGTFSIADAITDIQTIQQPYMTSIPPLNTTDINSTNPSASINCPGCQVHDGFQKAYRETMVNVQDRLVDFLGNNTDYKLIVTGHSLGAVTALFMGINLKNLGYDPTLINYGQPRLGNKAFADYVDALFFKQGDDGLTINPERRMYRVTHWNDFFVGWPAGYSHTLGEVYISDPTGINAPIEDVYSCAGPENNQCHHGSFNLLERLNILKNHCGYLNWIFYCAINVDKREMMIDPPRVGKRVEHWSGKFSDVESTEGLMYEAIYPM

SEQ ID NO: 12 - DNA, 1351 bp, Yarrowia lipolytica
agtgagtatggagagatggactggacttccatcatcgagaagatgtcacaggattgtctgttggcaggaagtctacagctagttgtggtacagtaccattggaagtggcggtggactattaagagttgaagttgttgatgtttctgagaatgcttatcgatcttggaaacgttcaccactgtcagcttatgctcagaacaactcaatgacaacctttccaactcagtacctcagttaattatctcctttgctaatttcagtgctctcgttcattgccctctttcttcttgttgctcaggctctcgcgcaaacggctcctatcacccaggagacatacgactttgttctcaagtacggatggctatctaacgtggcctattgtgtcagagcccctggcccatttgccctgcagagcgattttacctgtggaaactcttgcgctcattttcccgatgtgactcttgactaccagtttggaggaaatttcttctcgacttcagttactggatttttggcccacgatcacaccaaaaaggaaaagtatattgttttccgaggcactttctccattgcagacgctattactgacatccaaacgatccaacaaccctacatgaccagtatccctcctcttaacactactgacataaactccacaaacccttctgccagtatcaattgtcctggctgccaagttcatgatggctttcagaaggcatacagagagactatggtgaacgtacaggatcgtctggtcgacttccttggaaacaacactgactacaaattgatcgtaactggccactctctaggagctgtcactgctctattcatgggcatcaacctgaagaatcttggatatgatccaacgctaatcaactatgggcagcctcgacttggaaacaaggcttttgcagactacgtggacgctctgttctttaagcaaggagacgacggacttaccatcaaccccgaaagacggatgtaccgagtcactcactggaacgacttctttgttggatggcccgctggttattcacatactctgggagaagtttacatcagtgatcccacaggtattaatgctcctattgaagatgtctattcctgcgctggaccagagaataatcagtgtcaccatggatcattcaatcttctagaacgactcaacattctcaagaaccattgcggttatctcaattggattttctactgtgctatcaatgtggacaagagagagatgatgattgatcctccccgagttggcaagagagttgagcattggagtggcaagttttctgatgttgaatcgacagaaggcctgatgtatgaggccatttatcctatgtaa

SEQ ID NO: 13 - protein, 376 aa, Yarrowia lipolytica
MIVLLLLLSVALSSPIMLDRFGDSVNRMIRGNSEAYESGETRGVSQEFEQRLVRYMWFNNVAPCVPKKLQHPFKCIARGCKELGKHTELVDIFTHSDNLFDRTISGFVALDHKHKEIVLALRGTQDAHDWVTDLHLRLVGLHPEHLGVSNFNCRNCQVDLGFLKGYLHSFHVVDSIVQRLTEKYPDYQLVITGHSLGGTAATLFGLNYRLNGYSPLVFSAGAPALGNKQFANFADRVFWGSQNPNTLKVKERDIKFCRMTHLGDFVPRFPFWNGYQQMSGEVFINDVRGIYPPRETLQRCNGQQNRQCSFGDQYRKLEMNFKPHSAYLVPGSECSLSGRRELTFGQVHAANDTNKDSDTNIEPDIEPILIPVVQID

SEQ ID NO: 14 - DNA, 1131 bp, Yarrowia lipolytica
atgattgttctacttcttcttctctctgtcgcgttaagtagtccaattatgttggaccggtttggcgatagtgtgaaccgcatgatacgaggcaacagtgaggcgtatgagagcggcgagacacgcggggtgtcccaggagtttgaacagcggctcgtgcggtacatgtggttcaacaatgtggctccatgtgtccccaagaaacttcagcatccattcaagtgcatagctagaggctgcaaagagctggggaaacatacagagttggtcgacattttcactcattcagacaacttgtttgatcgcacaatttcaggatttgtggctttggatcacaagcacaaggagattgttctggcgttgagaggcactcaggatgcccacgactgggtgacagacctgcatctgaggctagtggggcttcatcccgagcatctgggtgtatcaaacttcaactgtcgcaactgccaggtagacctgggctttctcaagggatatttgcattcgttccatgttgttgacagtattgttcagcgtctgactgaaaaataccccgactaccaactggtgattacgggccattcacttggaggcactgcagctacgctctttggactcaactaccgactcaacggctattctcccctagttttttcggctggagctccggcactgggaaacaagcagtttgccaactttgccgaccgagtcttctggggatcgcagaaccccaacactctcaaggtcaaggagagagatatcaagttctgtcgcatgacccatctgggtgactttgtgcctcggtttcccttctggaacgggtaccagcaaatgtctggagaggtatttatcaacgacgtgcgaggtatatatccacccagggagacactccagcgatgcaacgggcaacagaatagacagtgctcgttcggagaccagtaccgcaagctggaaatgaacttcaagccccactcggcgtacttggtgcctggaagcgagtgttcgctcagtggtaggagagagttgacattcggacaagttcatgcagcgaatgacacaaacaaggactcagacacaaacattgagcctgacattgaaccaatcttgatacctgtcgtgcaaatagactaa

SEQ ID NO: 15 - protein, 406 aa, Yarrowia lipolytica
MAGFNFTFGQVISYLASMLYGQVDATSSSTRIQATQDLYDFTAKFSRLSNIAYCINAPFTPLRTDFTCGESCRYFPDLQLDSVFGGNFSSASTTGYIAYDHKKKEKYIVFRGTFSIPDIITDIQFQTAPWLTSLPTHLIPTKEDFEHKQAILKHYAAENKGLSNLEERQDVVHEDPSLVPKKMDKCENCQIHDGFAKGFNETIEHAGPQIEKFLGNNTDYKMFVVGHSLGAAQAQLFATQFKLLGFDPYMINFGQPRLGNPEFAAYINQLWFNDTGLVVNDARRFYRVTHWNDIVVGVPDWLNYTHSIGEVFIDEESVYPKLDKVVVCEGGENPLCHRGTFNLWSRINFLQNHLAYIFYIGLCAFNIGRRDVLNMPQYQGNFSYQHNIDPNYNYDTKVPTRISKSN

SEQ ID NO: 16 - DNA, 1221 bp, Yarrowia lipolytica
atggcagggttcaacttcactttcgggcaggtgatttcgtacctggcttccatgctctatggacaggtggatgccacttcatcctccactcgaatccaggccacccaggatctgtacgacttcacagccaagttttcgcgactctcaaacatcgcgtactgcatcaatgcccccttcacgcctctcagaacggacttcacctgcggagaaagctgtcggtacttccccgacctccagctggactcagtgtttggtggtaacttctcctcagcctccactaccggctacattgcatacgaccacaagaagaaggaaaagtacattgtgtttcgaggaactttcagtatccctgatatcatcacagacattcaatttcagactgccccttggttgacctctctgcccacgcatctgatccctaccaaggaggactttgaacacaagcaggccatcctgaagcactacgctgccgaaaacaagggtctcagcaacctggaagagcgacaggatgttgtgcatgaagaccctagcctggttcccaagaaaatggacaagtgcgagaactgccagatccatgacggattcgccaagggcttcaacgagactatcgagcatgccggaccccagattgaaaagttcctgggcaataacaccgactacaagatgtttgttgttggccactctctaggagctgctcaggcccagctgtttgctacacagttcaaactgctgggatttgacccttacatgatcaactttggacagcctcgacttggaaaccctgagttcgccgcctacatcaaccagctgtggttcaacgatactggtctggttgtcaatgatgcccgacgattctaccgagtgactcactggaacgatatcgtcgtgggagtgcccgactggctcaattacacccactctatcggagaggtgttcatagacgaggagagcgtttaccccaagctggacaaggtggtggtgtgcgagggaggagagaaccccctgtgccaccgaggaactttcaacctgtggtcacgaatcaacttcctgcagaaccatttggcttatatcttctacattggtctgtgtgctttcaacattggccgaagagacgtgctcaacatgccacaataccagggcaacttctcgtaccagcacaacatcgaccccaactacaattacgataccaaggttcccacccggatcagtaaatcaaactaa

SEQ ID NO: 17 - protein, 380 aa, Yarrowia lipolytica
MFGSTANSHNHMQLSPRSAGPYKSDYIYTDTYTPITTRLSQLSLRTMESLVEARKTIGKWALTSFFEGLSAAGKLHPQSDPKKHGVEVVSNIRYRETSPPQKSHYHLLDVYRPIKRDGPLPVVLYVHGGAFRILSKDTHWIMALAFARRGYVVANINYRLAPERPFPAALEDSCYAFKWVIDNIADYGGDVSQLVLAGESAGANLVTALSICATYARPEPYAQMVFNTNVAPKVVLPACGVLQVSDVDRFARQKDIPTWVVDRLEEVAFGYIGRKATANVGDRDTELANPLLILESDVEPVRPLPAYFATCGTKDVLIYDTKRLIPALEKHGAVAEAIYYPGEIHAFHALLFRKAAKKCWVDQLRFTKRILNKDKEKAKL

SEQ ID NO: 18 - DNA, 1143 bp, Yarrowia lipolytica
atgtttggctcaacagctaactcgcacaatcatatgcagttatctccgcgttctgcaggtccttataaaagtgactacatatatacagacacgtacactcccatcaccaccaggctatctcagctcagcttgcgcaccatggaatcactcgtcgaggctcgaaaaacaattggaaaatgggcgctgacgtccttctttgagggtctgtcggctgctggaaaactgcaccctcagagtgatcccaaaaagcacggagtggaggttgtgagtaacatccggtaccgcgagacctcgccaccgcaaaagtcgcactaccacctgctggacgtctaccgacccatcaagcgagacggacctctacccgtggttctctatgtccacggaggagcctttcgaattctctcaaaggacacgcactggatcatggccctggcgtttgcccgaagaggatacgtggtggccaacattaactatcgactggctcccgaacgcccctttcccgccgccctcgaggactcgtgctacgcctttaagtgggtcatcgacaacattgcagactacggaggagacgtgtcacagctggtgctggctggagaatcggctggagccaatcttgtgactgctctgtccatctgtgccacctacgcgcgacctgaaccgtacgctcagatggtcttcaacaccaacgttgctcccaaagtcgttcttcccgcatgtggagtgctgcaagtgtcagacgtggaccggtttgcacgtcagaaggatatccccacctgggtcgtggaccgactcgaagaggtggcgtttggatacattggacgcaaggccacagctaacgtcggagacagggacacagaattggccaacccgctattgattcttgagtcggacgtggaaccagtacgacccctgcctgcgtactttgccacctgtggaacgaaggatgttcttatctacgacaccaagcggctcatccccgcactggagaagcatggagcagttgcagaggccatctattaccccggagagatccacgccttccatgccttgttgtttagaaaggccgcaaagaaatgttgggttgaccagctgcgtttcaccaagcgaattctcaacaaggacaaggagaaggcaaagttgtag

SEQ ID NO: 19 - protein, 429 aa, Yarrowia lipolytica
MKASSMCSNQINQLAGFILDLHLSIFVLLMLQYSSETFLNRVVEGWSGAKGPENPTCKVIKRSTRSRRNSPESWTVSFSFFFASSFCSFSCFAMLLSSLVLSALSLVSVTAQNVVQATQDTWDLINYAEHLSSLAICGEPYGVIYKPFQCAGRCSDFPDMELITQFTPQDPLDFSVSGFLAVDHKRKVFWHVFRGTATLNNGLTDLRIKRQPLTSWNTAKMDCPDCQVHVGFLQAYNLAYSEAKGAMDDTFAKYPDYQVIVTGHSLGGAATFLHGINLKTSGYDPLVITSGQPLTGNKALADYNDKLFFGDNPDFTHQGPDRRFYRVTHKEDIVPRIPFWTPYHQSGGEVYIDFPGINPPVNTLKVCDGQQNPLCSFSTSLASTATQGIVEAAHLIYFTFFFLCSTLLYPPLNSDLPVGVWGKPLNGTI

SEQ ID NO: 20 - DNA, 1290 bp, Yarrowia lipolytica
atgaaagcttctagcatgtgttcaaatcaaataaatcaacttgccgggtttattctcgacctccacctgagtatatttgtacttctaatgttacagtatagtagtgagacttttttgaacagggtcgtggaggggtggagtggagcgaaagggcctgagaaccccacgtgtaaagtcataaaacggagtacaagaagtcgaagaaattccccagagtcctggaccgtcagcttcagcttcttcttcgccagctctttctgctctttctcctgcttcgccatgcttctctcctctcttgtcctttcagctctctctctggtgtctgtgacagctcagaacgttgtccaggccactcaggacacctgggacctgatcaactacgccgagcatctttcaagccttgcaatctgcggtgagccgtatggtgttatctacaaaccgttccagtgtgccggtagatgctccgacttccccgacatggagctcatcacccaattcaccccccaggatccgttggatttttcagtatcgggttttctggcagtagaccacaagcgaaaggtcttctggcatgtgttccggggcacggctacgctcaacaacggcctcactgacctaagaattaagcgacagcccctaacatcctggaacacggctaagatggactgtcctgattgccaagtgcacgttggctttctacaggcctacaatctggcctatagtgaggcaaagggcgctatggacgacacgttcgccaagtatcctgactatcaggtcattgtcactggccactctctcgggggagcagctactttcctgcacggaatcaacctcaagacctctggatacgaccctctggttatcacgtctggtcagccactcaccggtaacaaggctctggctgattacaacgacaagctcttctttggagacaaccccgattttactcaccagggtcctgaccgacgattctaccgagtcacccacaaggaagacattgtgcctcgaatccccttctggaccccctatcaccagagcggaggcgaggtgtacattgacttccccggaatcaaccctcctgtcaacaccctcaaggtctgtgacggccagcagaatcctctgtgttccttctccacctcactcgcctccactgccacccagggtatcgtcgaggccgcccatctcatttactttaccttctttttcctctgtagtacccttctctaccctcctctcaactccgatcttcctgtcggagtttggggtaagcctttgaacggcaccatttga

SEQ ID NO: 21 - protein, 324 aa, Yarrowia lipolytica
MRPSITIVGFLIASAVALGITQASKDTYNLLNYAENLNGVAVCVEQIGGIQKPFKCLGHCDDFPDMELITTFHPKKLFDFSTTGFLAIDHKRKQFWHVFRGTASLTDGISNLRLERQPLVFWDNPEFDCPGCEAHEGFLTAYNDAYDQIRDVLNQTLAQYPDYQIIVTGHSFGGASSFLHGINLKSQGMDPLVITSGQPLTGNKALADFNDKLFFGDNPDFTYQGPDRRFYRVTHKDDLVPRLPFWNPFHHSGGEVYIDYPLTNPPLRTLKICDGQQNPRCSFSTSLITAALLGTLQQAHFMYFTFFSLVCGVNIAGHLGPPLQ

SEQ ID NO: 22 - DNA, 975 bp, Yarrowia lipolytica
atgcgtccttctatcactatcgttggttttttaattgcctctgcagtcgccctaggtatcactcaagcttcaaaagatacctacaatctcttgaactatgccgaaaacctgaacggtgttgccgtttgtgttgagcagatcggcggaatccaaaagccatttaaatgtctgggtcattgtgacgacttccctgatatggagctaatcaccacatttcatccaaagaagctgtttgatttctctaccacaggatttttggctattgatcacaagagaaagcagttctggcatgttttcaggggtacggcctcactcacggatggaatatcgaacctgagactggagagacaacctctggtcttttgggacaatcctgagtttgactgtcccggttgtgaagcccatgaagggtttctgacggcctataacgatgcctatgatcaaatcagagatgtcctcaaccagactttggcccagtatccagactatcaaatcatcgttacaggccattcttttggtggagcgtcatcctttctccatggaatcaatctcaagagtcaaggcatggatccgttggtaatcacatctggacagccactcactggtaacaaggccttggcagacttcaatgataaactcttctttggagacaatccagattttacctaccaggggccagatcgacgattctatagagtcacccacaaggatgacctggtccctcgtcttcccttttggaacccctttcaccacagtggaggagaagtttacattgattatcctctaactaaccctcccctacggaccctcaagatttgcgatggtcaacagaaccccagatgctccttctccacctctcttataacagcagccctgttaggtaccctgcagcaagctcatttcatgtacttcacgtttttctccctagtctgcggtgttaatatcgctggccatcttggacctcctttgcagtga

SEQ ID NO: 23 - protein, 476 aa, Yarrowia lipolytica
MTTINSKALNGSFEGVPIGETIQWKNVLYADVKHRFAPPILKTDFEGKTIDCTEDGYDCPQLPNKHDMHDGEYKSDELLCTNLRIVKPKGDFKHPLPVYLYIHGGANLVGNIYSKRTDPTPFVEHSAAIGKPVIMVVIEYRLGMLGYSTSKDGKGNWGLRDQWTSVQWVNKFIGEFGGDSQRVTVGGESSGSIGVHALVLKDGLEHHGVISRAFMSSGSLGCFLPLPVAYLDTYRAKAAKELGVEEADLDDPTKVPPYALVTAGFKIEYPFGFYAYDDFLPDNLLQAIPKLRTALVSDNDYEGSLFVKRVVPPAEIEPLLNSSETGKKVKDIYSILTGAEVCKFRGDSFFVVSNAELLGQLEKGGVDVYRQFFDQVDPFDPSLRARHAVDLVFMWKCLPNLPEWAIELANKYQTNLIKFVYDESPWPSDEVALVGNKTIKYGHYNPRKLAEKLLDLDLKEIRKLGENLTPAGEWAM

SEQ ID NO: 24 - DNA, 1431 bp, Yarrowia lipolytica
atgactacaattaactcgaaagcactcaatggctcttttgagggagtccccattggggagaccatccagtggaagaacgttctgtacgccgatgtcaagcaccggtttgctcctcctattctcaagactgactttgagggaaagaccatcgactgtaccgaagacggatacgattgtccccagctgcccaacaaacacgatatgcacgatggcgagtacaagagcgacgagcttctgtgcaccaacctgcggatcgtgaagcccaagggcgacttcaagcacccccttcccgtctacttgtacatccacggaggtgccaacttggttggaaacatttacagcaagcgaaccgatcctactccgtttgtggagcactctgctgcaatcggcaagcctgtcatcatggttgtcattgagtatcgactcggtatgcttggttactccacctccaaggatggaaagggtaactggggtctgcgagaccagtggacttcagttcagtgggttaacaagttcattggtgaatttggaggagactctcagcgagtgactgttggaggagagtcttctggctcaattggtgtccacgctcttgttctgaaggacggcctcgagcaccatggcgttatcagccgagccttcatgtcttccggatctctgggctgtttccttcctcttccggttgcttatcttgatacttaccgagccaaggctgctaaggagcttggtgttgaagaggcggatctcgatgaccctaccaaggtccctccctacgctctcgtcacggccggattcaagatcgaatacccctttggcttctatgcttacgatgactttttgcctgacaacctgctgcaagccattcccaagctcagaaccgctcttgtctccgacaacgactatgagggttctctatttgtgaagcgagtggtgccacctgccgagatcgaacctcttctcaactcctccgagaccggaaagaaggtcaaggatatctacagcatcttgaccggtgccgaggtgtgcaagttccgaggagatagtttctttgtcgtgtctaacgcagagctactcggccaactggaaaagggaggtgttgacgtctaccgacagttcttcgaccaggttgatccctttgacccttctcttagggcccgtcatgctgtcgatcttgtcttcatgtggaaatgtcttcccaacctccctgagtgggccatcgaactggccaacaagtaccagaccaacctgatcaagttcgtctacgatgagtctccctggccctccgatgaggttgcccttgttggaaacaagaccatcaagtacggtcattacaaccctcgaaagctggcagagaagctgcttgacctcgacctcaaggagattcgaaagcttggagagaatctcactcctgctggagagtgggctatgtag

SEQ ID NO: 25 - protein, 486 aa, Yarrowia lipolytica
MSVTSTSLNGTFNGISEDGIEIFKGIKYANIPYRWAYAERIDDYDNGVFDCTQEGMACPQVLPFDYNIEKGPKEMPFDEFECSNLMITRPQGATNLPVFVWIHGGGNLAGNGYCSDHNPVPFVKHSIVAGRPVLHVMIEYRLSAFGYLAVPDTNGNWVGNWGARDQYTALQWISKHIVEFGGDPSQITIGGESAGSIGLHALMVHESMKPKEECIIHNVILSSGTMDRMGTGTISENAFKPIYDGIKTLVGDINTCSADELLEAQIKAGLDLGFYLQDDFFPPDWRNVRFKVSRVLLSDVIVDGTNFKNKINPAVRVTPENDFDHKVFKLYNISTEDTWEDYHYKMMLFKGDETFIRGNQQLELLFEQENIPVWRQLFDQIHPNDPSRLCHHAVDLYYMWDNWEMPEDKHAVARQYQDTLTKFVYGQDPWPVDKLHYVHDNQFEILDKSQFGDFRNVPALKFLLGFSAEELGELTKKYTGEGHYTL

SEQ ID NO: 26 - DNA, 1461 bp, Yarrowia lipolytica
atgtctgtcacatcaacttctctcaatggaaccttcaacggtatctctgaagacggtatcgagatcttcaagggtatcaagtacgcaaacattccgtatcgatgggcgtatgccgagcgaatcgacgactacgataatggcgttttcgactgcactcaggaaggcatggcttgtccgcaagtacttcctttcgactacaacattgaaaaggggcccaaggagatgccttttgatgagtttgaatgctccaatctcatgatcactcgaccccaaggcgctacaaatcttcctgtgtttgtatggatccacggaggaggaaatctggctggaaatgggtactgttccgatcacaaccccgttcctttcgtcaagcactccattgtggctggaagacccgttcttcatgtgatgatcgagtaccgtctcagtgcctttgggtatcttgctgttccggacacaaatggcaactgggttggcaattggggtgcccgagaccaatacacagccttgcaatggatctccaaacatatcgtggagtttggaggcgacccttcccagatcaccattggaggagagtccgcaggaagtattggacttcacgctctcatggtgcacgagagtatgaagcccaaggaagagtgtattatccacaatgttatcctctcgtcagggaccatggatcgaatgggtacggggacaatttcagagaatgcttttaagcccatctatgacggtatcaaaactctggttggagatatcaacacctgttctgcggacgagctgttggaggctcaaatcaaagccggcctcgacctcggcttttacctccaagacgacttcttccctccagactggagaaacgtgcggttcaaagtctctcgggttttactctcggacgtgattgtggatggaaccaacttcaagaacaagatcaatccagcggtacgagtaactccagagaacgactttgaccacaaggtcttcaagctctacaacatatctaccgaagatacatgggaggactaccactataagatgatgctatttaagggagacgagacctttattcgtggaaaccagcagctggaacttctgtttgagcaggaaaatatccccgtctggagacagttgttcgaccagattcatccaaatgaccccagtcgtctctgccaccatgccgtggatctctattacatgtgggacaactgggagatgcctgaagataaacatgcagtggcccgtcagtaccaggacactctgaccaagtttgtttacggacaagacccctggccggtcgacaagctccattatgtccacgataaccagtttgagatcctagacaagtctcagtttggcgacttccgcaacgttccagccctcaagtttctcttgggattctcagccgaagagcttggagaactcacgaaaaagtacactggagagggtcactacacgttgtag

SEQ ID NO: 27 - protein, 406 aa, Yarrowia lipolytica
MTFTIGIMNLTLGQVVAYLYASLFGEPPATLSKTRVQASQELYNFTAKFSRIANIAYCVNAPVTPLRTDFTCGESCRYFPNMTLDSVFGGDFYSTSITGYIAYDHLNKEKYVVIRGTFSIPDAVTDIQFQQSPWLVELPKHLIPTQDDMKQRWAVRPDLEVENKGLDNLKERALVVEDPRLIPIKTSECKGCMIHDGFAKAFNETMVNAAPQFEKFLTNHTDYKMYVTGHSLGAAQALLFATHFKLLGHDPTMINFGQPRVGNSEFANYINQLWFNDTGLEVNDKRRNYRLTHWNDIFVGLPDWSNYTHAIGEVYIDQESVYPALDKVSVCEGGENEACHRGTFNLWSRIDLLQNHLAYIYYIGYCALNIGRRNVLAMPKYHGNNSYKYATEPGYTGPGRTLVPTN

SEQ ID NO: 28 - DNA, 1221 bp, Yarrowia lipolytica
atgacattcactattggcatcatgaacctcacgctcgggcaagtggttgcgtacctctatgcctccctcttcggcgagccaccagcgacactttcaaaaactcgagttcaagcctcccaggagctgtacaactttacggccaagttttctcggattgcaaacatcgcctactgtgtcaatgcgccagtcactcctctacgaactgattttacatgtggagagagttgtcggtatttcccgaacatgacgttggactcagtcttcggaggagacttctattccacttcaatcaccggatacatagcttatgatcacctcaacaaggagaaatatgttgttattcgaggaacattcagcattccagatgctgttaccgacatccagtttcaacagtctccgtggttggtcgagcttcctaagcacctgatccctacccaagacgatatgaaacagagatgggcagtccgaccagacttggaagtggagaacaaaggtcttgacaacctcaaagagcgggctctggtggttgaagacccccgtcttattcccatcaaaactagtgagtgcaaaggctgcatgatccatgatgggttcgccaaggccttcaacgaaactatggtcaatgctgctcctcagtttgaaaagttcctcaccaaccacactgactacaaaatgtatgttactggtcactcgctgggcgctgctcaagctcttcttttcgcaactcacttcaaattacttggacatgatcctaccatgatcaactttggccagccgagagtaggaaactcagagtttgcaaactacatcaaccagctgtggtttaatgacacaggccttgaagtaaacgacaagcgacgaaactacaggctcacccactggaacgatatcttcgttggactgccagattggtccaattacacacacgccattggagaagtgtacattgaccaagaaagcgtctaccctgcactcgacaaagtgtctgtctgtgaaggaggagaaaacgaggcctgccatcgaggcactttcaatctgtggtctcgaatcgacctgctccaaaaccatctggcttacatctactacatcggctattgtgcacttaacattggtcggcgaaatgtcttagccatgcccaaataccacgggaacaactcctacaagtatgccactgaacctggttatacaggacccggtcgtacactggttcccaccaactaa

SEQ ID NO: 29 - protein, 624 aa, Yarrowia lipolytica
MIDEIFGRPSAKFKRVQCFAVALAWWLIITRGNKHGPPGIRSLSRALTAKITAWQIYVLTLTGYYLLKNFDKLIGLTPPIPFENHYTPGFYRATWVMQALDAGFWTAYPIKNKYARDLLSIVLTVYYLIFSDAANEKVRRYRLKTTVESCRVSWEKGRNPYAGAIAKLLLPKINIYKDVFVKRPAHSEYEEPVECLLLFDGTAEQLKNQKKVIYHLHGGGFTTMEPKLHEDYLLAWATQLKVPILSVNYRKAPEFPYPYALDEVFDTYYALMKSKGTIIGLSGDVVPEVVVTGDSAGGNLAAALELKLIDYPKKIKRPAGLVLTYPALDMNFASALSEDQMKLIRQVTKETATPFLYKNKKDVYQSYAGDAHKSVRANKSFVNLAQQQKVQSQQPGSPSEQGPGNKLSLTSKVLYSGDRVLPAEVLYTALMLYAGDTECDLSKDYLMSPLLAPEEKLAQFPKTYIICGEVDPLVDDTVLFGAKLRQAFRDSGRKNPEDLVEVSIIKGYSHGFLNMVPFFPEAKAPIHKLGFWMEEIFDNVLPEERNIDLNEDDYSTDEELVGESYANIDEEGGDVKAPRKKKTSAQKAWDKTNIVHPGGLVEGRQDKTKNSVIGEIKNAVVEKL

SEQ ID NO: 30 - DNA, 1875 bp, Yarrowia lipolytica
atgattgacgaaatctttggacgccccagtgcgaaattcaagcgggtccagtgctttgccgtggcgctggcctggtggctcatcatcacccgaggaaacaaacatggccctcccggaatccgatctctgagtcgagcgctcaccgccaaaatcaccgcgtggcaaatctacgtgctcacgctcacgggctactacctgctcaagaactttgacaagctcattggcctcacgccccccatcccattcgagaaccactacacccccggcttctaccgggccacatgggtgatgcaagcgctggacgcgggcttctggaccgcctaccccatcaagaacaagtacgcccgagacctgctgtccatcgtactcaccgtctactacctcatcttctcggacgctgccaacgagaaggtccgacgatacagactcaagaccaccgtggaatcgtgccgggtgtcatgggaaaagggccgaaacccgtacgccggcgccattgccaagctgctgttgcccaaaatcaacatctacaaggacgtgtttgtcaagcgacctgcccattccgagtatgaggagcccgtggagtgtctgcttctgtttgacggaactgccgagcagctcaagaaccagaagaaggtcatctaccatctgcacggaggaggattcaccaccatggagcccaagctgcacgaggactatctgcttgcctgggcaacccagctcaaggtgcccatcctctcggtcaactacagaaaggcccccgagtttccctacccttacgctctggacgaggtctttgacacctactacgcgctcatgaagtccaagggcaccatcatcggtctcagtggcgatgtcgtgcctgaggttgttgttaccggagactctgccggaggaaatctcgccgctgctctggagctcaagctcattgattaccccaagaagatcaagcgacccgctggtctggtgctcacctaccctgctcttgatatgaactttgcctctgctctgtccgaggaccagatgaagctcattcgacaggtgaccaaggaaactgccaccccgttcctctacaagaataagaaggacgtgtaccagtcatacgctggagatgcacacaagtctgttcgagccaacaagtcgtttgttaacctggctcagcagcaaaaggtccagtcgcagcagcctggttctccttccgaacagggtcctggaaacaaactgtctcttacttccaaggtgctttactccggagatcgagttctcccagccgaggttttgtacactgctctgatgctgtacgctggagacaccgagtgtgatctgtccaaggactatttaatgtccccccttctcgcacccgaagagaagcttgcacagttccccaagacgtacatcatctgtggtgaggtggatcctctggtggatgacactgttctgtttggagccaagctgcgacaagccttcagggactccggtagaaagaaccccgaggacctagtcgaggtatccatcatcaagggctactcccatggcttcctcaacatggtgcccttcttccccgaagccaaggctcccatccacaagctgggattctggatggaggagattttcgacaatgtgctgcccgaggaacgaaacatcgacctcaacgaggacgactacagcactgacgaggagctggtgggcgagtcgtacgccaacattgacgaagagggaggagatgtcaaggcccctagaaagaagaagactagcgcccagaaggcctgggataagaccaacattgtccatcctggaggactagtcgagggccggcaggacaagaccaagaattcggtcattggagagattaagaatgcggtcgtggagaagttgtaa

SEQ ID NO: 31 - protein, 327 aa, Yarrowia lipolytica
MLAMRPIRHRLGPNLLRPLHTTSLRLSEPRIIENDFATLRDKYNAPKWPVVLCHGLCGFNEIDTHLPVLPTYEYWGGIKESLEGAGATVITTAVPPVSSIKERAETLAGSLTQILHDAPWKKDTEKKSQQDKEEPKVNLIAHSMGGLDSRYLIHNLAKDLPFKVASLTTIATPHHGTSFADWCFDELGDKNVDRLYTILETLGVQNPDGFKQLTTTYLKEFNKTTQNDPDVAYYSYGAQYFPHWYNVFYPSWKIIDHYEGPSDGLVSVKSAQWGTYLGTINNCDHLDLINWTNRIRYHWYRLSGQRPSFNALAFYLHICDGLAKRGF

SEQ ID NO: 32 - DNA, 984 bp, Yarrowia lipolytica
atgctcgccatgaggccgatacgacacaggctggggccgaacctactccgaccactgcacacgacctcgctgcgtctctcggaaccccgaatcatagagaacgattttgccactctcagagacaaatacaatgctccaaaatggccagtggttctgtgccatggactgtgtggatttaatgagatcgacactcatctgcccgtgcttccaacatacgagtactggggaggcatcaaggagagtctggagggtgcaggagctacagtcatcaccacagcggtgcctccagtgagcagtatcaaggagcgggctgaaacgcttgctgggtcgctcactcaaattttgcatgatgctccttggaaaaaggacacggagaagaagagccagcaggacaaggaagagcccaaagtgaatctaattgctcacagcatgggcgggctggattcgcggtatttgatccacaacctagccaaagacctacccttcaaagtagcctctctcaccactatagctactcctcatcatggaacctctttcgcagactggtgctttgatgaacttggagacaagaatgtggaccggctatacaccattctggagactctaggagtccagaaccccgacggattcaaacagttgactaccacgtacttgaaggagtttaacaagaccacccagaacgatcctgatgtcgcttactactcatatggtgcgcagtacttccctcactggtacaatgtcttctacccttcctggaagatcattgatcactatgaaggtcctagtgatggattggtgagtgtcaagtccgctcagtggggtacttatctcggtacaatcaacaactgcgaccatctggatctcatcaactggaccaaccgaatacggtaccattggtatcgactttcaggccagagacccagtttcaatgctctggcattttatttgcatatttgtgatggtttagcaaagaggggtttctaa

SEQ ID NO: 33 - DNA, 13224 bp, Artificial Sequence
  plasmid MB8388 containing Hh/hdv, snr52
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgtgaagagcatttgcggccgcatagctcttctgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgttggaccccgtcttcaattacacttcccaactgggaacacccctctttatcgacccattttaggtaatttaccctagcccattgtctccataaggaatattaccctaacccacagtccagggtgcccaggtccttctttggccaaattttaacttcggtcctatggcacagcggtagcgcgtgagattgcaaatcttaaggtcccgagttcgaatctcggtgggacctagttgaaaaatacctctaatgcgccgatggtttagtggtaaaatccatcgttgccatcgatgggcccccggttcgattccgggtcggcgcaggttgacgttgaagagcatttgcggccgcatagctcttcgttttagagctagaaatagcaagttaaaataaggctagtccgttatcaacttgaaaaagtggcaccgagtcggtgcttttttttttcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 34 - DNA, 11606 bp, Artificial Sequence
  plasmid MB7452 expressing Cas9
cgcgtggatcgccggtgcgttgacgttggtgacctccagccagaggtgcccggcgccccgctcgccggcgaactccgtcgcgagccccatcaacgcgcgcccgaccccgtgcccccggtgctccggggcgacctcgatgtcctcgacggtcagccggcggttccacgccgagtacgagatgaccacgaagcccgccaggtcgccgtcgtccccgtacgcgacgaacgtccgggagtccgggtcgccgtcctccccgtcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctagaggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatggccgccagtgtgctggaattgaatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgttaggggcagggcatgctcatgtagagcgcctgccgctcgccgtccgaggcggtgccgtcgtacagggcggtgtccaggccgcagagggtgaaccccatccgccggta

SEQ ID NO: 35 - DNA, 7187 bp, Artificial Sequence
  plasmid
aagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagcgcggccgcacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcatctagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagg

SEQ ID NO: 36 - DNA, 66 bp, Artificial Sequence
  Ku70-d-Top-66 primer
atgacgagtctgatgagtccgtgaggacgaaacgagtaagctcgtcactcgtctacgcatgcctgg

SEQ ID NO: 37 - DNA, 66 bp, Artificial Sequence
  Ku70-d-Bot-66 primer
aacccaggcatgcgtagacgagtgacgagcttactcgtttcgtcctcacggactcatcagactcgt

SEQ ID NO: 38 - DNA, 24 bp, Artificial Sequence
  Ku70-c-Top-24 primer
cgtcgaacgtcttgtcttccgaag

SEQ ID NO: 39 - DNA, 24 bp, Artificial Sequence
  Ku70-c-Bot-24 primer
aaacttcggaagacaagacgttcg

SEQ ID NO: 40 - DNA, 66 bp, Artificial Sequence
  ura3-Cas9-Top-66 primer
atgttccttctgatgagtccgtgaggacgaaacgagtaagctcgtcaaggaacttgctcttaagca

SEQ ID NO: 41 - DNA, 66 bp, Artificial Sequence
  ura3-Cas9-Bot-66 primer
aactgcttaagagcaagttccttgacgagcttactcgtttcgtcctcacggactcatcagaaggaa

SEQ ID NO: 42 - DNA, 24 bp, Artificial Sequence
  ura3-2-Top-24 primer
cgtgatatcataattgtcggccgg

SEQ ID NO: 43 - DNA, 24 bp, Artificial Sequence
  ura3-2-Bot-24 primer
aaaccggccgacaattatgatatc

SEQ ID NO: 44 - DNA, 22 bp, Artificial Sequence
  lip16_pcr_rev_full primer
cgccaaatggtaccatttttgg

SEQ ID NO: 45 - DNA, 18 bp, Artificial Sequence
  lip16_pcr_for_full primer
ctgccactcctcctgctc

SEQ ID NO: 46 - DNA, 60 bp, Artificial Sequence
  Lip16Dbtm primer
gactagctcgatatgagaagtgaaggtgaaggtgaaggtgagcgggagcggtctgagaaa

SEQ ID NO: 47 - DNA, 60 bp, Artificial Sequence
  Lip16Dtop primer
cctgcttgtcgttcatcgaaattgtgtcgttgctccaagttttctcagaccgctcccgct

SEQ ID NO: 48 - DNA, 24 bp, Artificial Sequence
  LIP17-24-Bot primer
aaaccagttaattatctcctttgc

SEQ ID NO: 49 - DNA, 24 bp, Artificial Sequence
  LIP17-24-Top primer
cgtgcaaaggagataattaactgg

SEQ ID NO: 50 - DNA, 21 bp, Artificial Sequence
  LIP18 rev seq primer
caagtgaatggcccgtaatca

SEQ ID NO: 51 - DNA, 21 bp, Artificial Sequence
  LIP18 for seq primer
tggtgaccgctacaatccaac

SEQ ID NO: 52 - DNA, 19 bp, Artificial Sequence
  LIP18 rev pcr primer
gcgatccccagaagactcg

SEQ ID NO: 53 - DNA, 22 bp, Artificial Sequence
  LIP18 for pcr primer
acttgtactcctcttcctccca

SEQ ID NO: 54 - DNA, 24 bp, Artificial Sequence
  LIP3-Cas9-24-b-Bot primer
aaacctgagtgagttgaacaaggt

SEQ ID NO: 55 - DNA, 24 bp, Artificial Sequence
  LIP3-Cas9-24-b-Top primer
cgtaccttgttcaactcactcagg

SEQ ID NO: 56 - DNA, 34 bp, Artificial Sequence
  LIP8-Cas9-34-Bot primer
tctaaaactcgtgaattttgcagccctcacgtca

SEQ ID NO: 57 - DNA, 34 bp, Artificial Sequence
  LIP8-Cas9-34-Top primer
ggttgacgtgagggctgcaaaattcacgagtttt

SEQ ID NO: 58 - DNA, 24 bp, Artificial Sequence
  LIP18-Cas9-24-Bot primer
aaacggcagttgcgacagttgaag

SEQ ID NO: 59 - DNA, 24 bp, Artificial Sequence
  LIP18-Cas9-24-Top primer
cgtcttcaactgtcgcaactgccg

SEQ ID NO: 60 - DNA, 66 bp, Artificial Sequence
  LIP18-Cas9-66-Bot primer
aaccatgatacgaggcaacagtggacgagcttactcgtttcgtcctcacggactcatcagcactgt

SEQ ID NO: 61 - DNA, 66 bp, Artificial Sequence
  LIP18-Cas9-66-Top primer
atgacagtgctgatgagtccgtgaggacgaaacgagtaagctcgtccactgttgcctcgtatcatg

SEQ ID NO: 62 - DNA, 20 bp, Artificial Sequence
  LIP17 rev seq primer
gcgctcattttcccgatgtg

SEQ ID NO: 63 - DNA, 26 bp, Artificial Sequence
  LIP17 for seq primer
tcaatcatcatctctctcttgtccac

SEQ ID NO: 64 - DNA, 20 bp, Artificial Sequence
  LIP17 rev pcr primer
cctattgtgtcagagcccct

SEQ ID NO: 65 - DNA, 22 bp, Artificial Sequence
  LIP17 for pcr primer
aggccttctgtcgattcaacat

SEQ ID NO: 66 - DNA, 23 bp, Artificial Sequence
  LIP16 rev seq primer
gagaaaaagtcgcctccaaactg

SEQ ID NO: 67 - DNA, 21 bp, Artificial Sequence
  LIP16 for seq primer
tcggcatttgagagaccttgg

SEQ ID NO: 68 - DNA, 22 bp, Artificial Sequence
  LIP16 rev pcr primer
gagaaaaggtgcctcggaaaac

SEQ ID NO: 69 - DNA, 24 bp, Artificial Sequence
  LIP16 for pcr primer
tttaggttccgcattaaactctcg

SEQ ID NO: 70 - DNA, 24 bp, Artificial Sequence
  LIP17-Cas9-24-Bot primer
aaactacatgaccagtatccctcc

SEQ ID NO: 71 - DNA, 24 bp, Artificial Sequence
  LIP17-Cas9-24-Top primer
cgtggagggatactggtcatgtag

SEQ ID NO: 72 - DNA, 66 bp, Artificial Sequence
  LIP17-Cas9-66-Bot primer
aacacaggtattaatgctcctatgacgagcttactcgtttcgtcctcacggactcatcagatagga

SEQ ID NO: 73 - DNA, 66 bp, Artificial Sequence
  LIP17-Cas9-66-Top primer
atgtcctatctgatgagtccgtgaggacgaaacgagtaagctcgtcataggagcattaatacctgt

SEQ ID NO: 74 - DNA, 24 bp, Artificial Sequence
  LIP16-Cas9-24-Bot primer
aaactcgctcaaacagccaacatc

SEQ ID NO: 75 - DNA, 24 bp, Artificial Sequence
  LIP16-Cas9-24-Top primer
cgtgatgttggctgtttgagcgag

SEQ ID NO: 76 - DNA, 66 bp, Artificial Sequence
  LIP16-Cas9-66-Bot primer
aaccagggctgtattagaccaccgacgagcttactcgtttcgtcctcacggactcatcagggtggt

SEQ ID NO: 77 - DNA, 66 bp, Artificial Sequence
  LIP16-Cas9-66-Top primer
atgaccaccctgatgagtccgtgaggacgaaacgagtaagctcgtcggtggtctaatacagccctg

SEQ ID NO: 78 - DNA, 24 bp, Artificial Sequence
  Lip8 rev seq primer
aggaccagtgtaatgttcctgatc

SEQ ID NO: 79 - DNA, 20 bp, Artificial Sequence
  Lip8 for seq primer
agctgtctcctttcctggtc

SEQ ID NO: 80 - DNA, 20 bp, Artificial Sequence
  Lip8 rev pcr primer
agctgtctcctttcctggtc

SEQ ID NO: 81 - DNA, 35 bp, Artificial Sequence
  Lip8 for pcr primer
cacacacgcgtgaagaggatggtggaaagcttcat

SEQ ID NO: 82 - DNA, 31 bp, Artificial Sequence
  LIP2ioRevXba primer
cacactctagattgaaaacctcatggtggtg

SEQ ID NO: 83 - DNA, 35 bp, Artificial Sequence
  LIP2ioFwdMlu primer
cacacacgcgtagggtgtctgtggtatctaagcta

SEQ ID NO: 84 - DNA, 35 bp, Artificial Sequence; identical to SEQ ID NO: 81
  LIP2iorevMlu primer

SEQ ID NO: 85 - DNA, 35 bp, Artificial Sequence
  LIP2ioFwdkpn primer
cacacggtaccattatttgtttaagcgacaacttg

SEQ ID NO: 86 - DNA, 24 bp, Artificial Sequence
  LIP8-Cas9-24-Bot primer
aaacatcctcgttcagcctgctgt

SEQ ID NO: 87 - DNA, 24 bp, Artificial Sequence
  LIP8-Cas9-24-Top primer
cgtacagcaggctgaacgaggatg

SEQ ID NO: 88 - DNA, 66 bp, Artificial Sequence
  LIP8-Cas9-66-Bot primer
aactcgtgaattttgcagccctcgacgagcttactcgtttcgtcctcacggactcatcaggagggc

SEQ ID NO: 89 - DNA, 66 bp, Artificial Sequence
  LIP8-Cas9-66-Top primer
atggccctcctgatgagtccgtgaggacgaaacgagtaagctcgtcgagggctgcaaaattcacga

SEQ ID NO: 90 - DNA, 30 bp, Artificial Sequence
  LIP2 for seq primer
aagtagatagttgaggtagaagttgtaaag

SEQ ID NO: 91 - DNA, 18 bp, Artificial Sequence
  LIP2 rev pcr primer
tctggagaactgcggcct

SEQ ID NO: 92 - DNA, 28 bp, Artificial Sequence
  LIP2 for pcr primer
agaggaacgcatatacagtaatcataga

SEQ ID NO: 93 - DNA, 24 bp, Artificial Sequence
  LIP2-Cas9-24-Bot primer
aaaccgctgttgatcatgcctcca

SEQ ID NO: 94 - DNA, 24 bp, Artificial Sequence
  LIP2-Cas9-24-Top primer
cgttggaggcatgatcaacagcgg

SEQ ID NO: 95 - DNA, 66 bp, Artificial Sequence
  LIP2-Cas9-66-Bot primer
aaccggtgacgaagtactgcagagacgagcttactcgtttcgtcctcacggactcatcagtctgca

SEQ ID NO: 96 - DNA, 66 bp, Artificial Sequence
  LIP2-Cas9-66-Top primer
atgtgcagactgatgagtccgtgaggacgaaacgagtaagctcgtctctgcagtacttcgtcaccg

SEQ ID NO: 97 - DNA, 22 bp, Artificial Sequence
  LIP3 rev seq primer
ctttgaacgtgacattgaccgg

SEQ ID NO: 98 - DNA, 22 bp, Artificial Sequence
  LIP3 for seq - (really reverse) primer
acgtgtccagatattcatccgc

SEQ ID NO: 99 - DNA, 16 bp, Artificial Sequence
  LIP3 rev pcr primer
ggcaactggggcacgt

SEQ ID NO: 100 - DNA, 20 bp, Artificial Sequence
  LIP3 for pcr primer
ccgaatcacttgaacgcaca

SEQ ID NO: 101 - DNA, 24 bp, Artificial Sequence
  LIP3-Cas9-24-Bot primer
aaaccttgggaggctgaggagtga

SEQ ID NO: 102 - DNA, 24 bp, Artificial Sequence
  LIP3-Cas9-24-Top primer
cgttcactcctcagcctcccaagg

SEQ ID NO: 103 - DNA, 66 bp, Artificial Sequence
  LIP3-Cas9-66-Bot primer
aacctgagtgagttgaacaaggtgacgagcttactcgtttcgtcctcacggactcatcagaccttg

SEQ ID NO: 104 - DNA, 66 bp, Artificial Sequence
  LIP3-Cas9-66-Top primer
atgcaaggtctgatgagtccgtgaggacgaaacgagtaagctcgtcaccttgttcaactcactcag

SEQ ID NO: 105 - DNA, 25 bp, Artificial Sequence
  tglseq-rev primer
tacttggttcgtacatgacctatag

SEQ ID NO: 106 - DNA, 25 bp, Artificial Sequence
  tglseq-fwd primer
gcctatttcgggccctaagagaacc

SEQ ID NO: 107 - DNA, 60 bp, Artificial Sequence
  TglDelta-rev primer
caatctgttttcttctataataccattacctatatttcttaccaataccggggacgtgca

SEQ ID NO: 108 - DNA, 60 bp, Artificial Sequence
  TglDelta-fwd primer
accctctcctctgtcaacaaaccaccgacttataaaaagatgcacgtccccggtattggt

SEQ ID NO: 109 - DNA, 66 bp, Artificial Sequence
  Ku70-e-Bot-66 primer
aacatcgtcaatgttcctatatcgacgagcttactcgtttcgtcctcacggactcatcaggatata

SEQ ID NO: 110 - DNA, 66 bp, Artificial Sequence
  Ku70-e-Top-66 primer
atgtatatcctgatgagtccgtgaggacgaaacgagtaagctcgtcgatataggaacattgacgat

SEQ ID NO: 111 - DNA, 66 bp, Artificial Sequence; identical to SEQ ID NO: 40
  ku70RightseqFwd primer

SEQ ID NO: 112 - DNA, 26 bp, Artificial Sequence
  ku70LeftseqFwd primer
aacaatactacacaatccctttcctg

SEQ ID NO: 113 - DNA, 24 bp, Artificial Sequence
  Lip4-5′-top-24 primer
cgtggtggcctggattcgagtggg

SEQ ID NO: 114 - DNA, 24 bp, Artificial Sequence
  Lip4-5′-bot-24 primer
aaacccactcgaatccaggccacc

SEQ ID NO: 115 - DNA, 66 bp, Artificial Sequence
  Lip4-3′-top-66 primer
atggtgtaactgatgagtccgtgaggacgaaacgagtaagctcgtcttacacccactctatcggag

SEQ ID NO: 116 - DNA, 66 bp, Artificial Sequence
  Lip4-3′-bot-66 primer
aacctccgatagagtgggtgtaagacgagcttactcgtttcgtcctcacggactcatcagttacac

SEQ ID NO: 117 - DNA, 25 bp, Artificial Sequence
  LIP4-5′seq-fwd primer
tgcaggggtgtcggagtaggtatac

SEQ ID NO: 118 - DNA, 26 bp, Artificial Sequence
  LIP4-3′seq-rev primer
gtatatagaaacgaaaatcttgttag